(19) United States
(12) Patent Application Publication
(10) Pub. No.: US 2014/0155297 A1
(43) Pub. Date: Jun. 5, 2014

(54) METHOD AND APPARATUS FOR LIGHT BASED RECOVERY OF SEQUENCE VERIFIED DNA

(71) Applicant: CAMBRIAN GENOMICS, INC., San Francisco, CA (US)

(72) Inventor: Austen Heinz, San Francisco, CA (US)

(73) Assignee: CAMBRIAN GENOMICS, INC., San Francisco, CA (US)

(21) Appl. No.: 13/775,745

(22) Filed: Feb. 25, 2013

Related U.S. Application Data
(60) Provisional application No. 61/603,019, filed on Feb. 24, 2012.

(51) Int. Cl. C12N 15/10

(52) U.S. Cl. CPC C12N 15/1093
USPC 506/30; 506/36

(57) ABSTRACT

A method of retrieving sequence-verified deoxyribonucleic acid (DNA) from a DNA sequencing plate includes directing a laser beam to impinge on a predetermined area on the DNA sequencing plate. The DNA sequencing plate contains a plurality of sequence-verified DNA disposed thereon. The method also includes exposing the predetermined area to a radiation dosage provided by the laser beam to generate at least one of an optical pressure or an ablating force in the predetermined area. The optical pressure or ablating force result in the ejection of at least one sequence-verified DNA disposed proximate to the predetermined area.

### CROSS-REFERENCES TO RELATED APPLICATIONS

This application claims priority to U.S. Provisional Patent Application No. 61/603,019, filed on Feb. 24, 2012, the disclosure of which is hereby incorporated by reference in its entirety for all purposes.

### BACKGROUND OF THE INVENTION

Synthetic nucleic acids (e.g., synthetic DNA) have applications in molecular biology and biomedical research and development. The synthesis of synthetic nucleic acids is typically performed using column-based synthesizers.

The applications for synthetic nucleic acid polymers include use as primers for the polymerase chain reaction (PCR) and sequencing of DNA.

Despite the progress made in the synthesis and recovery of synthetic DNA, there is a need in the art for improved methods related to DNA synthesis and recovery.

### SUMMARY OF THE INVENTION

The present invention relates to the field of artificial DNA synthesis, DNA sequencing, and on a broader level, the recently established field of synthetic biology. Embodiments of the present invention can be used for the fabrication of genes, gene circuits, small mitochondrial chromosomes, large bacterial chromosomes, and the like. In particular, embodiments are relevant to whole genome modification such as codon optimization or alternate codon schemes for artificial amino acids or any other project related to large scale rewriting.

According to an embodiment of the present invention, a method of retrieving sequence-verified deoxyribonucleic acid (DNA) from a DNA sequencing plate is provided. The method includes directing a laser beam to impinge on a selected or predetermined area on the DNA sequencing plate. The DNA sequencing plate contains a plurality of sequence-verified DNA disposed thereon. The method also includes exposing the selected area to a radiation dosage provided by the laser beam to generate at least one of an optical pressure or an ablating force in the selected area, thereby ejecting at least one sequence-verified DNA disposed proximate to the selected area.

According to another embodiment of the present invention, a system for retrieving sequence-verified deoxyribonucleic acid (DNA) is provided. The system includes a laser source and a DNA sequencing plate containing the sequence-verified DNA disposed thereon. The system also includes optical elements disposed along an optical path between the laser source and the DNA sequencing plate and a collection substrate adjacent the DNA sequencing plate.

According to an embodiment of the present invention, a method for sequence verification is provided. The method includes positioning one or more oligonucleotides on a cover slip of a total internal reflection fluorescence microscope and illuminating the one or more oligonucleotides with an evanescent wave of an excitation light beam. The method also includes forming a fluorescence image of the one or more oligonucleotides by collecting an emission light beam emitted from the one or more oligonucleotides using an objective lens of the total internal reflection fluorescence microscope and observing the fluorescence image to verify the sequence of the one or more oligonucleotides.

In an embodiment, the total internal reflection fluorescence microscope is at least one of a cis or trans total internal reflection fluorescence microscope. As an example, the one or more oligonucleotides can be synthesized by using a parallel single molecule synthesis process. Moreover, at least one of the one or more oligonucleotides can have at least one fluorophore attached to a DMT group thereof.

According to another embodiment of the present invention, a method of synthesizing oligonucleotides is provided. The method includes providing a reagent fluid containing oligos to a microfluidic chamber. The microfluidic chamber includes a microfluidic chip positioned proximate to a cover slip of a total internal reflection fluorescence microscope, at least one working electrode disposed on the microfluidic chip, and at least one auxiliary electrode. The method also includes applying an electrical potential (e.g., a periodic wave such as a square waveform or a saw-tooth waveform) between the at least one working electrode and the at least one auxiliary electrode while observing a fluorescence image of the microfluidic chip using the total internal reflection fluorescence microscope, thereby adding nucleotides in selected locations on the microfluidic chip. The working electrode can include a transparent conducing oxide (TCO), such as indium tin oxide (ITO).

As an example, the at least one auxiliary electrode can be disposed on a same plane as the working electrode, for example, with the at least one auxiliary electrode including at least two auxiliary electrodes flanking the at least one working electrode on each side thereof. In another example, the at least one auxiliary electrode can be disposed on a plane that opposes the working electrode. Moreover, the at least one working electrode can include a plurality of working electrodes.

In some embodiments, the electrical potential is provided by a potentiostat coupled to the at least one working electrode and the at least one auxiliary electrode. The microfluidic chamber can also include a reference electrode coupled to the potentiostat. As examples, the reference electrode can include Ag, AgCl, Cu, or Cu(II).

According to a specific embodiment of the present invention, a method of retrieving DNA from a substrate is provided. The method includes directing a beam of light to a selected area on the substrate. The substrate includes one or more polonies or rolonies attached thereon via one or more photocleavable linkers. As an example, the one or more polonies or rolonies can be assembled by at least one of Gibson Isothermal assembly or yeast assembly of overlapping oligonucleotides released from a parallel DNA sequencing plate.

The method also includes exposing one or more photocleavable linkers in the selected area to a radiation dosage provided by the beam light to sever the one or more exposed photocleavable linkers, thereby separating one or more polony DNAs or rolony DNAs from the substrate. In one embodiment, the one or more photocleavable linkers are sensitive to the beam of light characterized by a wavelength range and the beam of light characterized by the wavelength range is not damaging to the one or more polonies or rolonies. As an example, directing the laser beam can include steering the laser beam using a digital micromirror device (DMD) or be performed using a liquid crystal display (LCD) mask. In some implementations, the substrate can include a electrokinetic concentrator.

Some embodiments of the present invention relate to single molecule synthesis using total internal reflection (TIRF) microscopy as well as single molecule assembly of oligonucleotides using electrophoretic forces and TIRF observation. Other embodiments relate to the assembly of colony DNA (many copies of the same oligonucleotide) using various light based DNA recovery methods to recover oligonucleotides from a massively parallel sequencing chip. This includes laser ejection of DNA using a continuous or pulsed laser. Laser ejection may use a fixed beam and dual stages or having a fixed sequencing chip and a moving collection mechanism and using beam steering techniques such as galvonometer mirrors or a rotating polygonal mirror. Additionally, embodiments of the present invention relate to another category of light devices for DNA capture from a massively parallel sequencer. This category of devices combine a light based release mechanism using light emitting diodes (LED), digital micromirror (DMD), or liquid crystal display (LCD), photoelectric release, or fixed mask, or other light based methods to release DNA to and from a massively parallel sequencer with a fluidic collection mechanism such as electrokinetic concentration.

Numerous benefits are achieved by way of the present invention over conventional techniques. For example, embodiments of the present invention provide improved methods and systems for characterizing and recovering synthetic DNA sequences. These and other embodiments of the invention along with many of its advantages and features are described in more detail in conjunction with the text below and attached figures.

### BRIEF DESCRIPTION OF THE DRAWINGS

FIG. 1 is a simplified diagram illustrating a conventional DNA synthesis method using standard acid based deprotection of the DMT group;

FIG. 2 is a simplified diagram of an inkjet based oligosynthesis setup;

FIG. 3 illustrates an electrode based mechanism for DNA synthesis;

FIG. 4 illustrates an iterative photochemical process whereby protected dNTPs are added to an oligo during DLP based oligo synthesis;

FIG. 5 illustrates the release of DNA from a electrode based DNA synthesis scheme;

FIG. 6 illustrates the amplification of substrate bound oligos using PCR;

FIG. 7 illustrates the oligonucleotide chemical synthesis using fluorophore bound DMT groups to allow total internal fluorescence microscopy observation of synthesis according to an embodiment of the invention;

FIG. 8 is a simplified diagram illustrating a possible TIRF microscopy setup for monitoring the incorporation of phosphoramidites onto an oligo in which the excitation and emission beam follow the same optical train according to an embodiment of the invention;

FIG. 9 is a simplified diagram illustrating an alternative TIRF setup in which the excitation beam travels through a quartz prism and the emission beam travels through the objective according to an embodiment of the invention;

FIG. 10 illustrates a strategy for optical observation of oligonucleotide assembly using fluorescently labeled oligonucleotides according to an embodiment of the invention;

FIG. 11 is simplified diagram illustrating a TIRF DNA observation unit in which electrophoresis is used to direct oligos to certain locations on the chip for assembly according to an embodiment of the invention;

FIG. 12A is a simplified diagram illustrating a TIRF oligo assembly observation setup in which two auxiliary electrodes are on each side of the working electrode according to an embodiment of the invention;

FIG. 12B is a simplified diagram illustrating a TIRF oligo assembly observation setup where multiple working electrodes are located on opposite planes according to an embodiment of the invention;

FIG. 12C a simplified diagram illustrating a TIRF electrophoretic setup in which a single working electrode is located opposite to the auxiliary electrode according to an embodiment of the invention;

FIG. 13 shows a pulse laser embodiment in which a top stage is coupled with a bottom collector stage according to an embodiment of the invention;

FIG. 14 a simplified diagram illustrating a galvanometer-scanning-mirror apparatus used for beam steering according to an embodiment of the invention;

FIG. 15 a simplified diagram illustrating laser ejection of DNA using a laser in addition to an LED for imaging and the use of spools for collection of ejected DNA onto flexible film according to an embodiment of the invention;

FIG. 16 a simplified diagram illustrating a laser scanning method using a polygonal mirror according to an embodiment of the invention;

FIG. 17 a simplified diagram illustrating a technique in which closely packed separated sequencing substrates are spread out over a larger area so that each substrate can align with a collection tube according to an embodiment of the invention;

FIG. 18 a simplified diagram illustrating redundancy in multiple sequencing lanes according to an embodiment of the invention;

FIG. 19 a simplified diagram illustrating the use of a barrier or ejection layer used to shield DNA from the heat laser cavitation/shockwave according to an embodiment of the invention;

FIG. 20 a simplified diagram illustrating the various forces and effects of pulse laser DNA ejection according to an embodiment of the invention;

FIG. 21 is a simplified diagram illustrating plasma treatment of a polymer collection film either to allow faster reagent loading through fluidic self-assembly or to ensure ejected DNA is captured in the appropriate collection area prior to liquid loading according to an embodiment of the invention;

FIG. 22 is a simplified diagram illustrating the digital micromirror setup for releasing polony/rolony DNA bound to a substrate via a photocleavable linker according to an embodiment of the invention;

FIG. 23 is a simplified diagram illustrating an alternative embodiment in which an LCD is used to control the position of light on the DNA wafer for programmatic photocleavage;

FIG. 24 is a simplified diagram illustrating a bead bound to a substrate by a photocleavable linker for DMD release according to an embodiment of the invention;

FIG. 25 is a simplified diagram illustrating fluorescent labeling of rolony DNA via hybridization of 5′ tagged fluorophore oligos for rolony visualization according to an embodiment of the invention;

FIG. 26 is a simplified diagram illustrating release and visual confirmation of rolony DMD release via photocleavable linker according to an embodiment of the invention;

FIG. 27 is a simplified diagram illustrating a grid approach to positional mapping and a patterned flow cell approach to achieving desired spacing according to an embodiment of the invention;

FIG. 28 is a simplified diagram illustrating an electrokinetic concentrator for collection of DNA released into a microfluidic channel according to an embodiment of the invention;

FIG. 29 is a simplified diagram illustrating a process of fabricating nanoporous charged material in a microfluidic channel for electrokinetic concentration according to an embodiment of the invention;

FIG. 30 is a simplified diagram illustrating release of rolony or bead based DNA into a microfluidic channel for trapping by an electrokinetic concentrator according to an embodiment of the invention;

FIG. 31 is a simplified flowchart illustrating a method of recovering sequence verified DNA according to an embodiment of the present invention; and

FIG. 32 is a simplified schematic diagram of a two-part flow cell according to an embodiment of the present invention.

### DETAILED DESCRIPTION OF SPECIFIC EMBODIMENTS

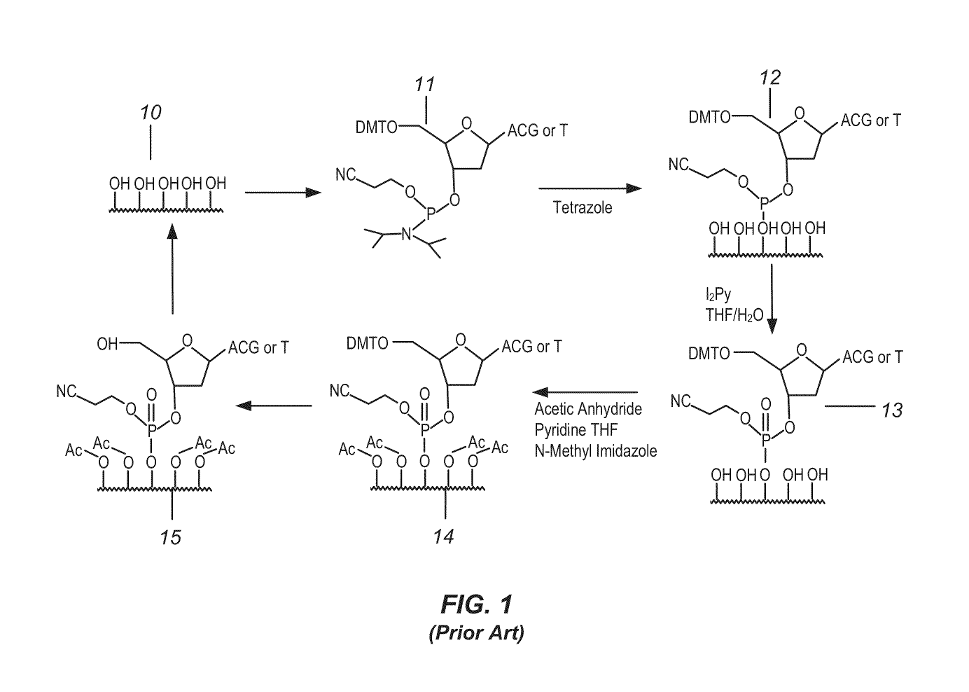

FIG. 1 is a simplified diagram illustrating a DNA synthesis process using a 10 hydroxyl linker surface and the addition of a nucleoside phosphoramidite 11. Tetrazole is added, allowing the coupling reaction to occur between the nucleoside phosphoramidite and the scaffold 12. To complete the coupling reaction THF/H₂O are added 13 to stabilize the phosphite triester internuceosidic linkage. Next a capping reaction is performed by the addition of acetic anhydride, pyridine THF, and N-Methyl Imidazole. After the completion of the coupling reaction, a small percentage of the solid support-bound 5′-OH groups remain unreacted and need to be blocked from further chain elongation to prevent the formation of oligonucleotides with an internal base deletion 14. Afterwards the next deprotection step is performed in which an acid is used to remove the DMT group, thereby starting the process over 15.

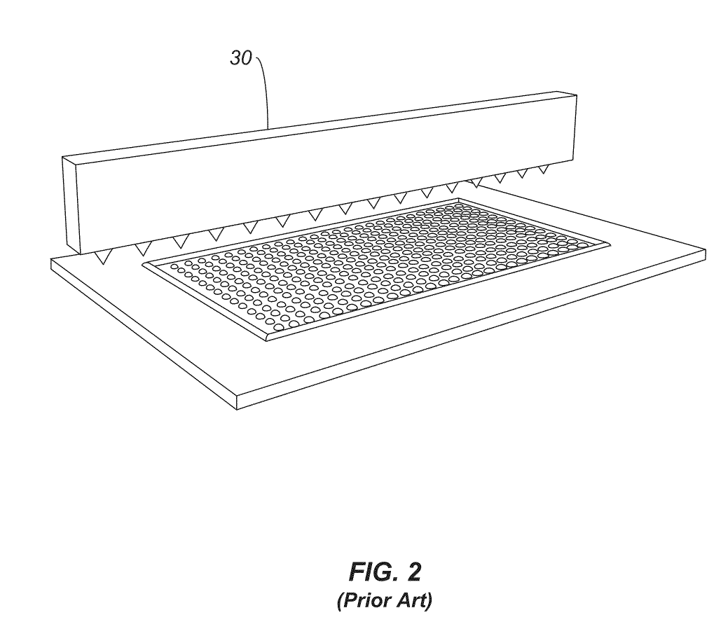

FIG. 2 is a simplified diagram illustrating a inkjet printer head 30 used for standard phosphoramidite chemistry. An open source inkjet printer design known as POSaM was published by Hood et al. in 2004 Inkjet printing of oligos within the same confinement has previously been used to assemble DNA into larger pieces. This strategy for assembling DNA does not have a sequencing step prior to the first round of assembly, requiring enzyme based error correction methods to lower error. Inkjet fabricated oligos are desirable because of the low error rates, which allow for the fabrication of long >150 bp sequences.

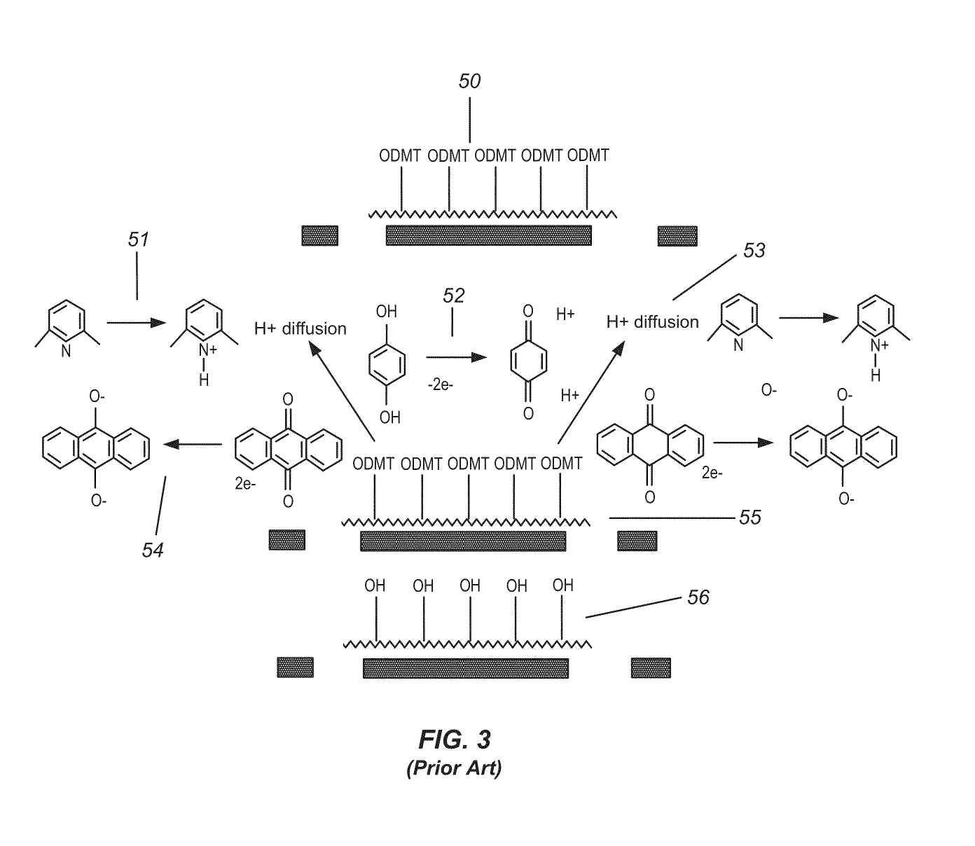

FIG. 3 is a simplified diagram illustrating the process of making DNA using electrode based deprotection. Circular platinum anodes approximately 100 um are surrounded by cathodes. In the region in immediate proximity to the anode an acidic environment is generated in response to a voltage set via connection to an external PC. Diphenylhydrazine 52 is used as the electrogenerated acid. Acid diffuses outside the electrode region which can result in cross-contamination. To contain this acid within isolated regions a base is included in the medium 51. The surrounding cathode activates a neutralizing base 53, which does not effect the DMT protection groups. The electrode is shown before 50, during 51, and after activation 52.

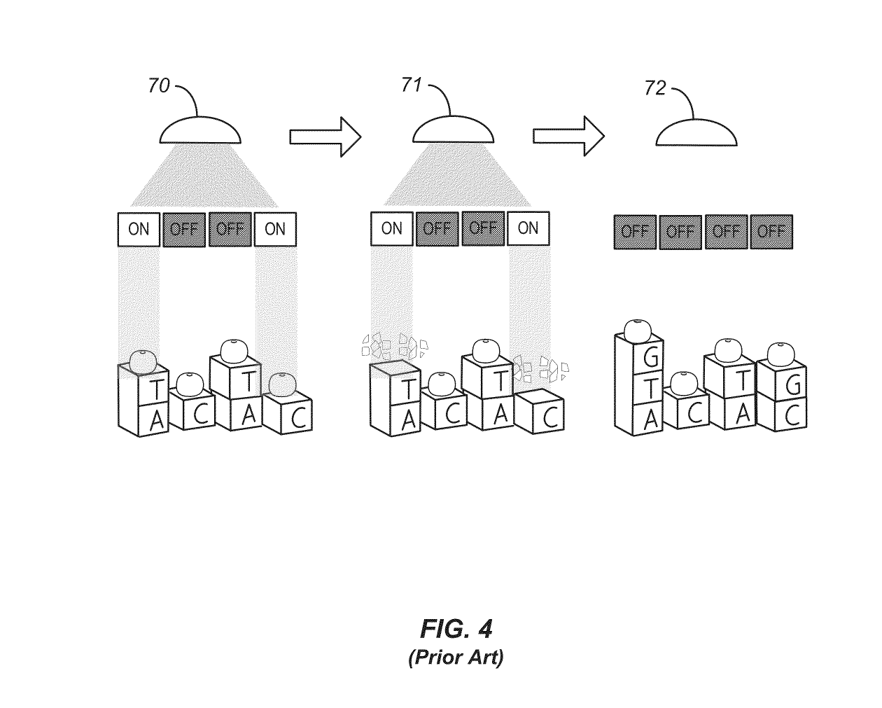

FIG. 4 is a simplified diagram illustrating a light based DNA synthesis. This illustration is designed to represent both NPPOC and standard phosphoramidite chemistry in which a photogenerated acid (PGA) such as CH₂Cl₂ is used for deprotection. When the oligos are hit with light 70 the protective group is removed 71 and next dNTP can be added (in this case G) 72. As with all synthesis approaches, a flow cell is necessary. There are several options here ranging from building customized individually automated valves to simply using a modified DNA synthesizer such as the ABI 391 as in the microfluidic array synthesizer (MarS) design¹⁰ or a Expedite 8909 DNA synthesizer in the photogenerated acid approach currently used by LC Sciences.

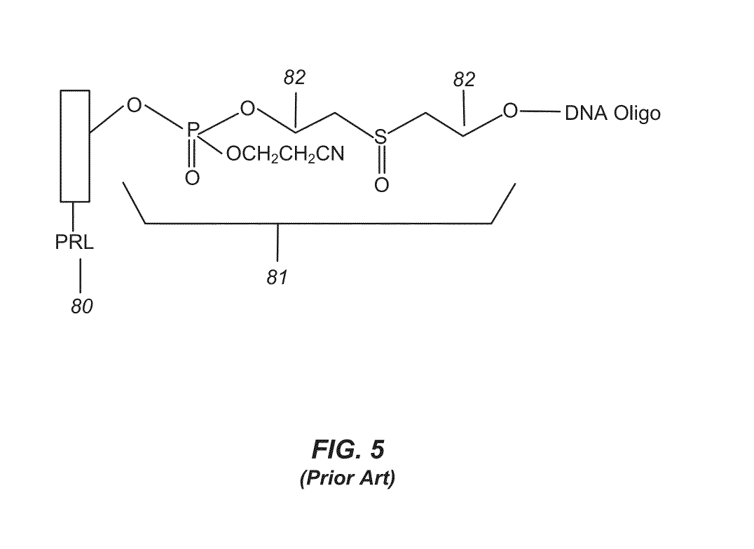

FIG. 5 is a simplified diagram illustrating a DNA attached to a semiconductor porous reaction layer (PRL) 80 which provides a solid support for attachment, similar to controlled pore glass CPG used in the column of a commercial synthesizer. Base cleavable linkers can be used to release the DNA from the substrate so that the DNA can be recovered. Unlike PCR based recovery shown in the next figure, cleaving the DNA from the substrate and directly sequencing does not introduce errors and can result in more stable stoichiometry during sample prep and sequencing. 81 shows the linker unit produced by a chemical phosphoralation reagent.

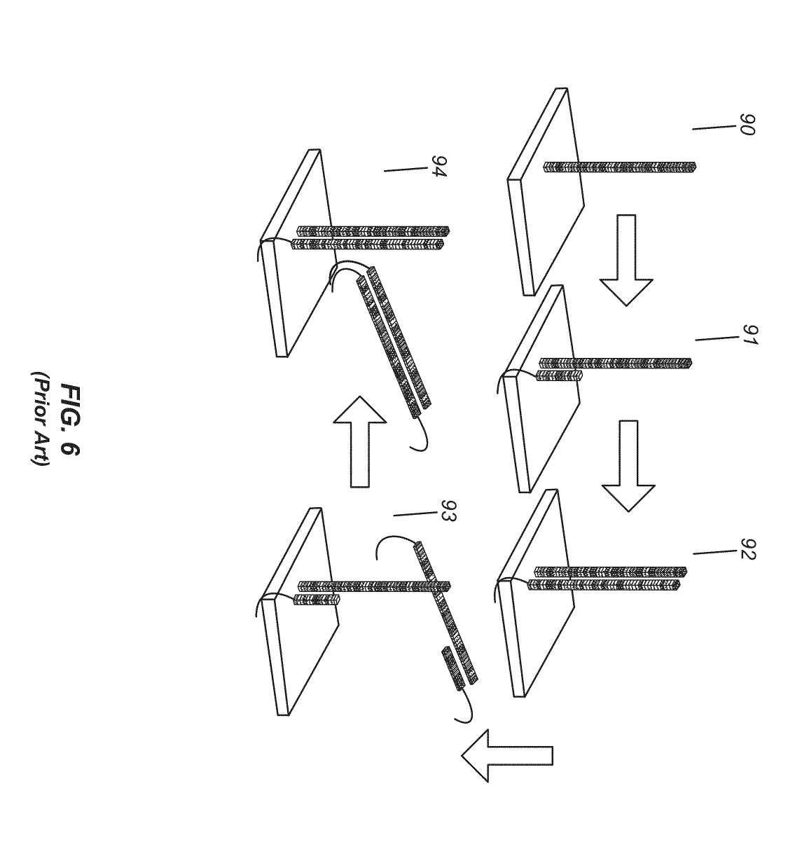

FIG. 6 is a simplified diagram illustrating the PCR amplification of substrate bound i.e. microarray DNA prior to primer binding 90, after primer binding 91, after extension 92, after extension of dehybidization and primer binding to both strands 93, and after extension of both strands 94. This approach has been used previously to provide DNA for subsequent assembly. Amplification can be done from both microarray manufacturers as shown and from free floating oligos that are provided from vendors.

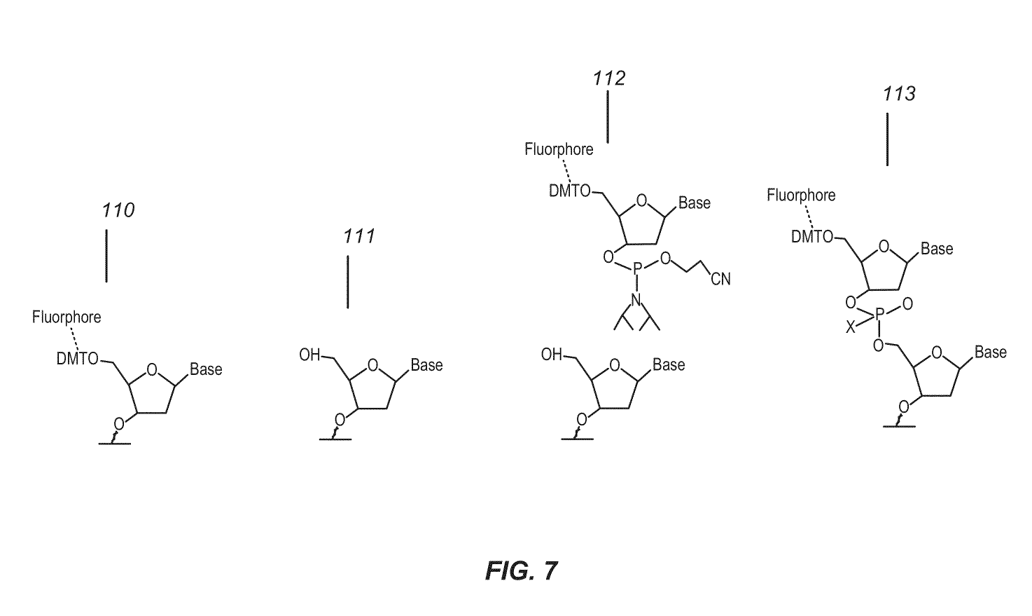

FIG. 7 is a simplified diagram illustrating the iterative single molecule chemical synthesis in which a protected base having a fluorophore attached to the DMT group of an oligo bound to a glass scaffold 110 and then de-protected (detritylation) 111. The deprotection event removes the fluorophore to allow for the visualization of the next oligonucleotide addition also known as coupling 112. Afterwards the fluorescent DMT capped oligonucleotide is capped and then undergoes oxidation (x=O) or undergoes sulfurization (x=S) followed by capping 113. The synthesis must be done at low enough density to allow for independent recovery of the single DNA molecules using optical tweezers, electrokinetic forces (electrophoresis, dielectrophoresis, travelling-wave dielectrophoresis), magnetic tweezers, acoustic traps, or optoelectronic tweezers. A patterned synthesis surface (not shown) may assist in allowing for separation between molecules for total internal reflection microscopy (TIRFM) observation and recovery.

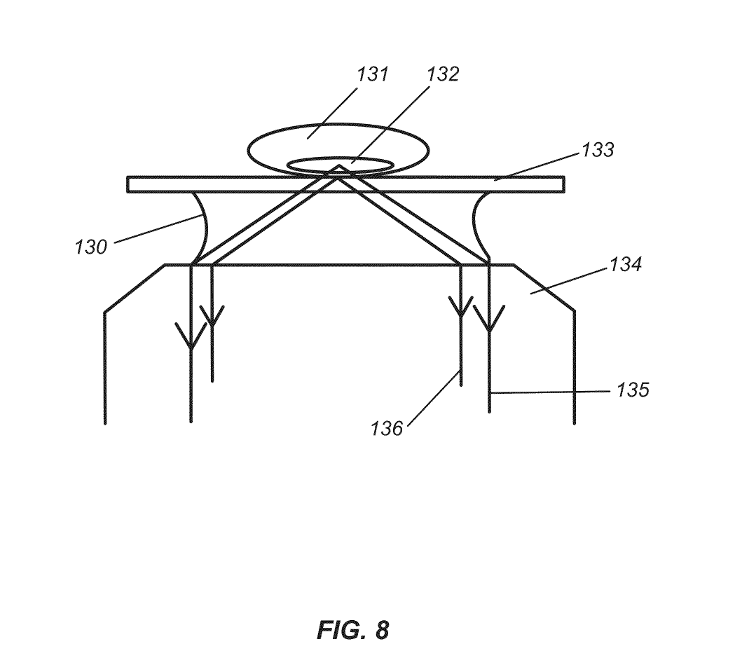

FIG. 8 is a simplified diagram illustrating a possible total internal reflection fluorescence microscope (TIRFM) system used to observe single molecule chemical synthesis using fluorophore linked DMT blocked oligonucleotides. TIRFM uses an evanescent wave—a near field standing wave that undergoes exponential decay—immediately adjacent to the glass water interface. This removes the background of unbound fluorescent nucleotides or other noise. In the TIRFM setup illustrated in FIG. 8, the observation solution 131 covers the synthesized oligonucleotides—which fall in the evanescent wave range of approximately 100 nm 132 attached to a glass substrate 133. The lens is covered with oil to increase the resolution of the microscope by increasing the refractive index and thus the numerical aperture of the objective lens 135. The excitation beam 136 and the imaging beam 135 are also shown.

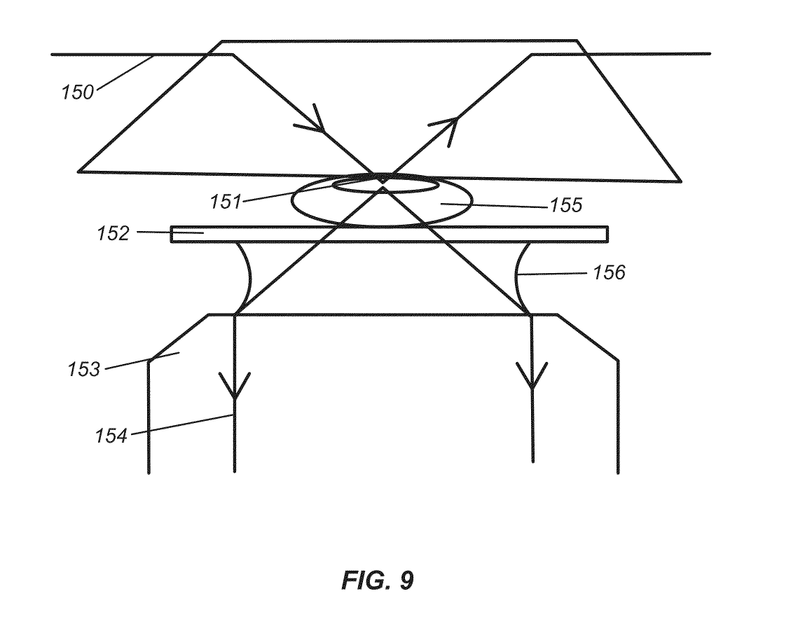

FIG. 9 is a simplified diagram illustrating an alternate optical arrangement in which the excitation beam 150 is illuminated through a quartz crystal on the fluorescence observed oligonucleotide synthesis region 151 in observation solution 155. Once again the emission beam 154 is directed through a cover slip 152 through the high refractive index oil medium 156 into the objective 153.

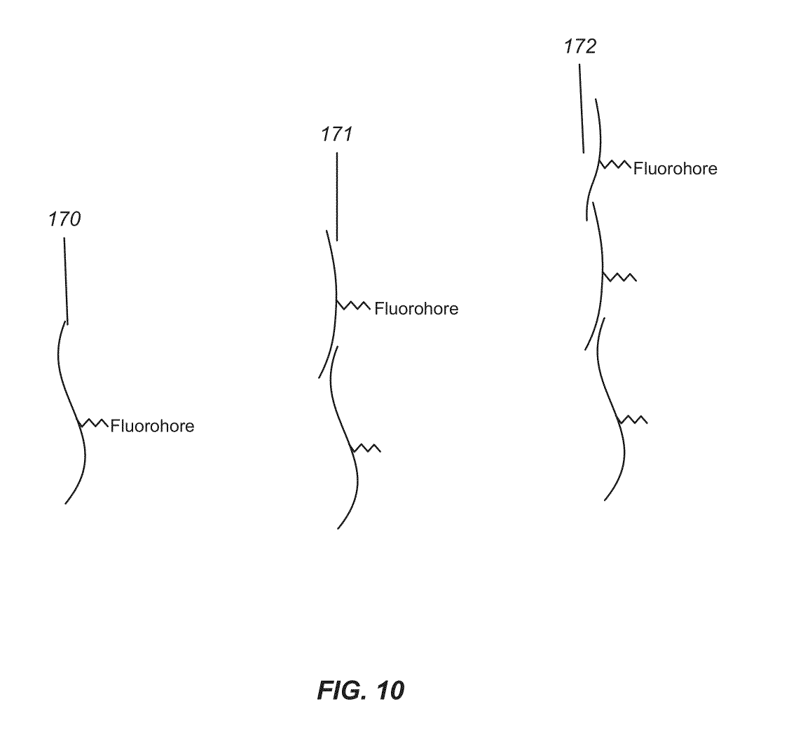

FIG. 10 is a simplified diagram illustrating single molecule assembly of oligos using a reversible fluorophore. Three step assembly shown 170, 171, 172. The fluorophore allows for TIRF based visualization of hybridization of oligonucleotides. Fluorescently labeled oligonucleotides may be targeted to locations on the chip using TIRF electrochemistry using eletrophoretic forces in a similar manner to Nanogen to create distinct electrophoretic fields. This can allow precise targeting of hybridizing polymers to intended sequences. The reversible fluorophore can be a single hybridizing base with attached fluorophore attached through hybridization to the non-overlapping sequences or an acid or photocleavable cleavable linker attached to the 5′ or 3′ end of the oligonucleotide.

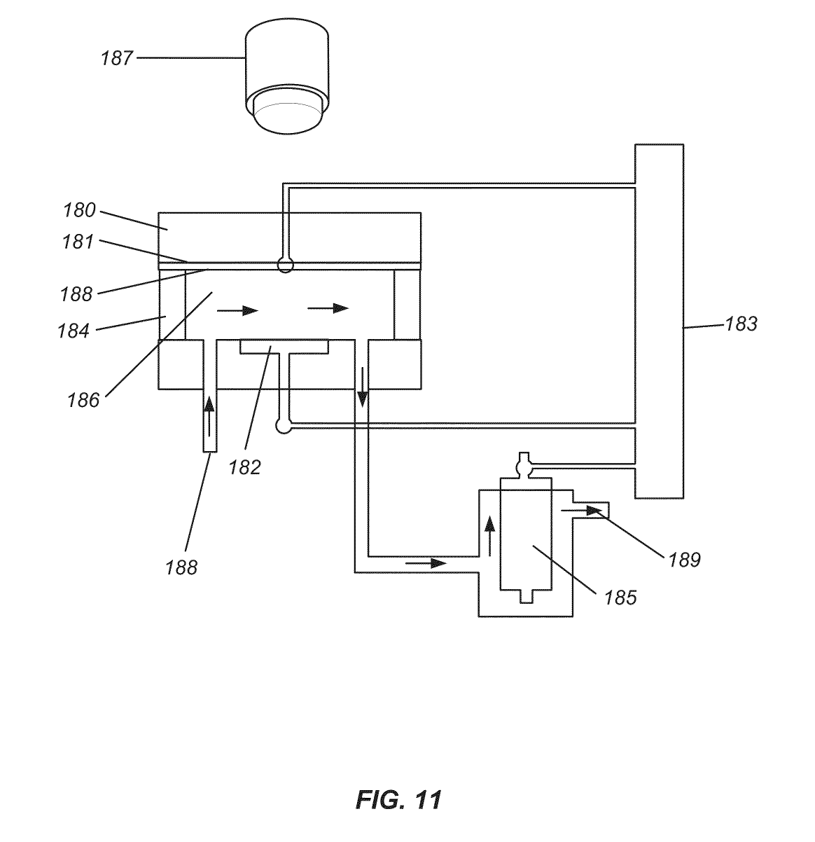

FIG. 11 is a simplified diagram illustrating a TIRF single molecule electrokinetic DNA assembly platform with a TIRF prism 180, and TIRF slide 181 covered by a transparent working ITO electrode 188 which working, a platinum electrode 182 auxiliary connected to a potentiostat 183. An Ag/AgCl or Cu/Cu(II) reference electrode is also connected to the potentiostat 185. A low conductance buffer 186 containing oligos to be assembled inside a microfluidic chamber 184. TIRF optics are located on the side of the working electrode 187. Fluidic input 188 and output 189 are also shown.

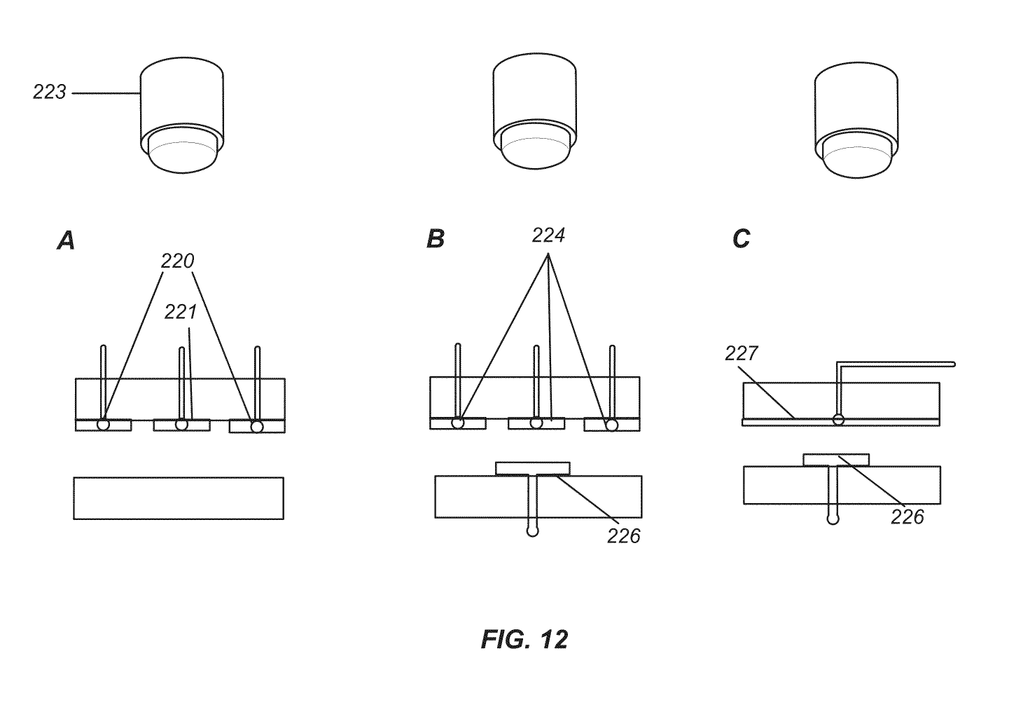

FIG. 12 is a simplified diagram illustrating various TIRF electrophoretic setups. 12A shows a working electrode 221 flanked by two auxiliary electrodes 220. TIRF optics are also shown 223. This design is useful for scaling since many of these electrodes can be fabricated in parallel using modern semiconductor fabrication to allow DNA molecules to be processed by their charge and hybridize to various on chip locations. 12B shows another design capable of scaling. Here several working electrodes 224 are positioned orthogonal to an auxiliary electrode 226. 12C is provided for comparison purposes. As in the previous figure a single working electrode 227 is positioned orthogonal to the auxiliary electrode 226. Altering the parameters of this electrode system are necessary to prototype and measure how electronic hybridization compares against passive hybridization as well as how electronic stringency can be used to improve the selectivity and discrimination between strands. Ionic strength, PH, and temperature are all parameter spaces that must be explored.

Although DNA synthesis, for example, on a microarray, has been developed, separation of the sequenced DNA is provided by embodiments of the present invention. In an embodiment, DNA is attached to a bead and then copied multiple time (e.g., 100,000 times) on the bead. The beads are then attached to a substrate (e.g., a glass surface). Parallel sequencing of the DNA attached to the beads is then performed to provide an atlas of the DNA beads correlated to position on the substrate. As an example, a bead ID, a coordinate position on the substrate, a readout of the sequence, and a quality score can be provided for each bead. Once the desired DNA are determined, for example, using the quality score, the laser ejection techniques described herein are utilized to recover the desired DNA.

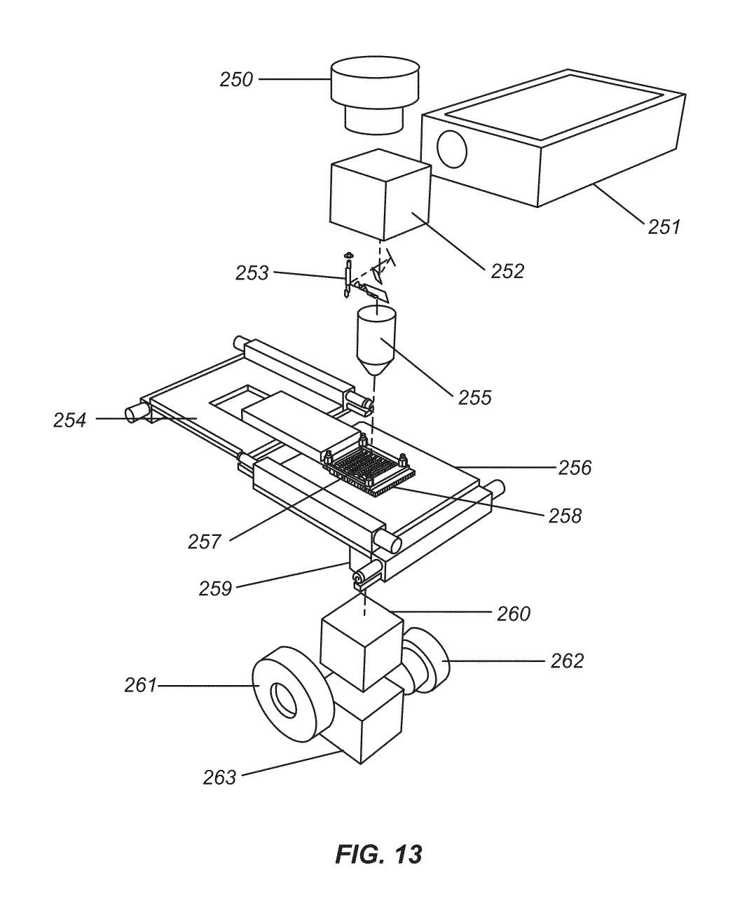

FIG. 13 is a simplified diagram illustrating a laser based release system in which clonal DNA on a parallel sequencer (e.g., a massively parallel sequencer) is ejected from the sequencing plate via the use of a laser. One of the obvious advantages of using a laser, as opposed to other optical methods such as optical tweezers, is that it is potentially much more rapid. Using a laser to eject substrate bound clonal DNA is also advantageous because it can be done in a dry environment at high throughput without risk of cross contamination. The mechanism behind clonal DNA ejection is through a combination of optical pressure (the momentum of photons) and the explosive ablative force resulting from energy absorption, which while generating significant local heating, is locally contained in the vicinity of the focal spot, which can be as small as a micron in diameter. Pulse lasers focused through an objective can create very high energy densities (e.g., up to 10 MW/cm² with a typical UVA pulse laser).

Embodiments of the present invention differ from the previous use of pulsed lasers to capture cells and even organelles for DNA recovery via PCR after collection into a tube. This has gone by several names such as laser capture microdissection, laser microdissection, laser assisted microdissection, and laser catapulting. Laser wavelengths in the UV-A range and above, such as a 337 nm wavelength produced by a nitrogen laser or a 355 nm wavelength produced by a solid state laser fall out of the absorption wavelength of proteins and DNA however as previously mentioned significant heating can occur, which can be biologically destructive. At low flux, material absorbs heat and can evaporate or sublimate, however at high flux, as is the case with a pulsed laser or powerful continuous beam laser, the flux can turn a material to plasma. This expanding plasma can be used to apply a pulse of high pressure to the surface underneath it. Taking advantage of this capacity, a protective polymer layer can be useful to eject the sample while limiting the amount of heat absorbed by or damage imparted on the sample.

Referring to FIG. 13, a pulsed laser 251 is directed onto a half mirror 252, where it is steered using galvonometer scanning mirrors 253 focused by an F-Theta objective lens 255. The F-Theta lens is used to create a flat planar imaging field. The F-theta lens is able to achieve this effect by adding a specific amount of barrel distortion to the scanning lens. After focusing, the beam is directed by the lens onto the DNA chip 257 located on the top stage 254 which ejects a sequenced DNA bead onto the 96/384/1536 well plate 258 on the bottom stage 256. A bottom objective 259 is also used below the collector stage in order to allow a CCD 262 to properly align the collector stage via the bottom half 260 and full mirror 263. Meanwhile the top CCD 250 is used to align the bead bound DNA for ejection into the collector plate. A light source at the bottom 261 is used to provide illumination. Although the laser is shown mounted above the stage, in some embodiments this arrangement is modified since it can be more convenient to place the laser on a table surface and use full reflecting mirrors to direct the beam upwards. One of ordinary skill in the art would recognize many variations, modifications, and alternatives.

As discussed above, an atlas is created during sequence verification, providing a database that lists individual sequence verified DNA as a function of position on the DNA chip. Using the atlas, it is possible to position the DNA chip 257 at a predetermined location and direct the laser beam produced using pulsed laser 251 to impinge on the position of the desired individual sequence verified DNA. Registration of the DNA chip to the laser system is utilized using mechanical, electromechanical, and/or optical systems to provide for accurate ejection of desired DNA. In an embodiment, a list of desired sequence verified DNA is provided. The atlas is used to determine the locations of the DNA in the list of desired sequence verified DNA. In a sequential manner, the pulsed laser is directed to impinge on the locations associated with the DNA in the list, sequentially removing the desired DNA from the DNA chip.

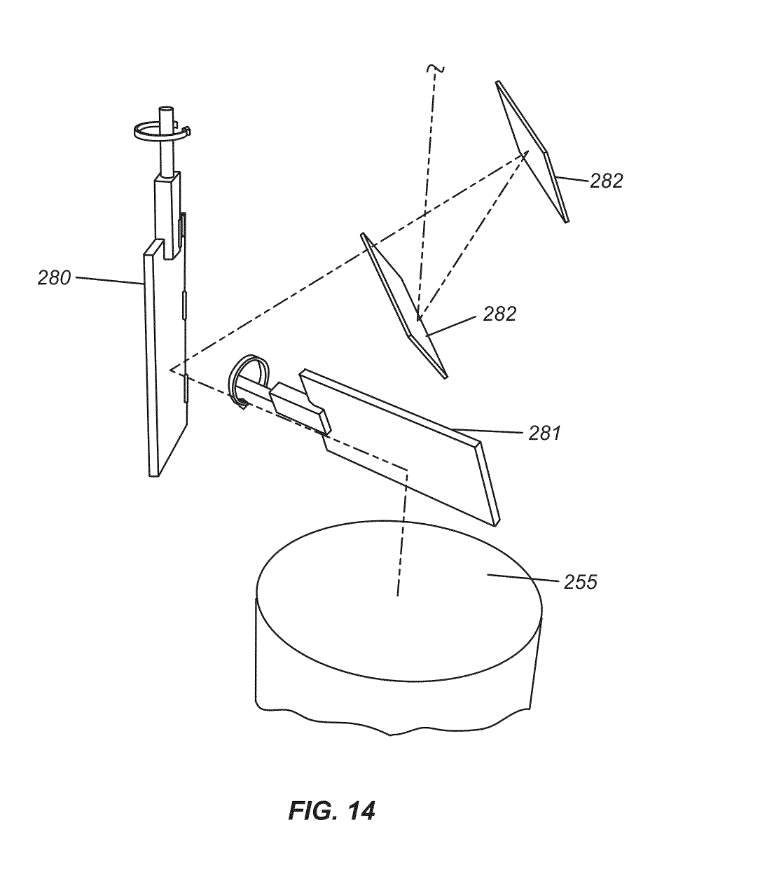

FIG. 14 is a simplified diagram illustrating a closer view of the beam steering apparatus known as a scanning galvanometer. Two fixed mirrors 282 redirect the beam from the source to the objective where it is directed onto the scanning mirrors. One of the mirrors scans the X axis the other scans the Y axis. In this system the top stage can move to several fixed positions and eject substrate or carrier bound DNA.

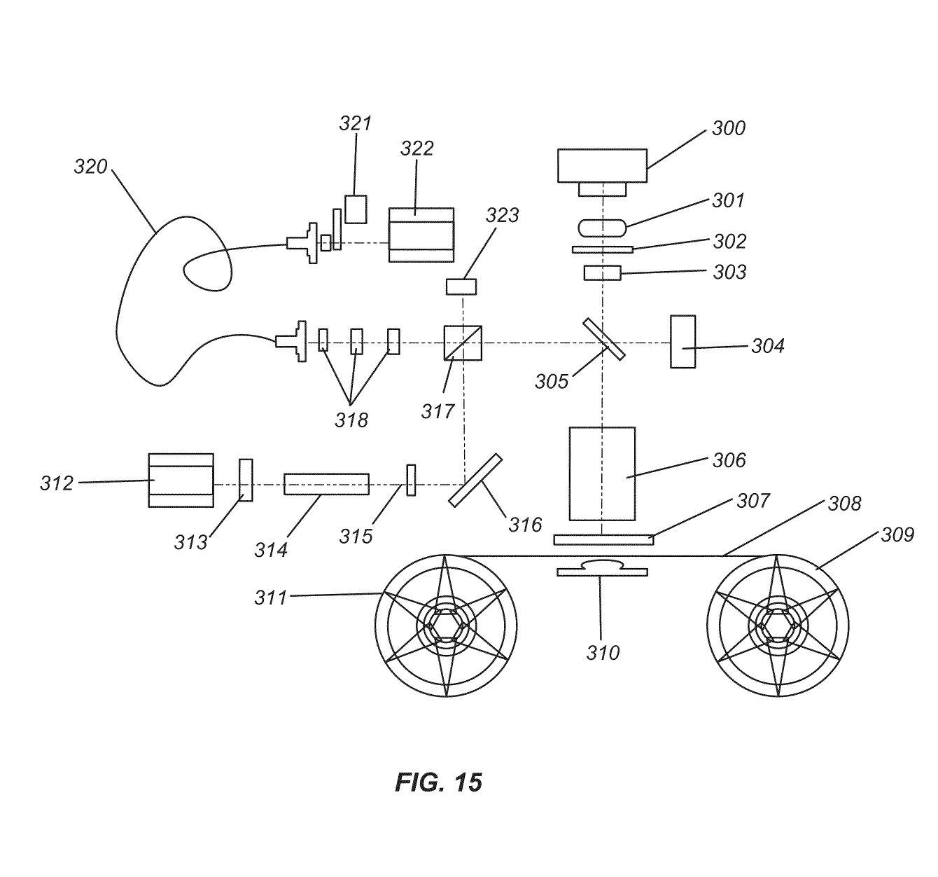

FIG. 15 is a simplified diagram illustrating a galvonometer based scanning DNA ejection system. In this configuration a flexible plastic film recovery device including tape 308 is utilized. The collector plastic can include microlitre wells and is either treated with corona discharge or is filled with reagent prior to ejection so that carrier bound clonal DNA finds its appropriate destination. Spindles 309 and 311 are used to move the collector plastic 308 underneath the laser beam impingement spot. Not shown are liquid loading after the loading spindle 311 or liquid sealing prior to the right spindle 309. In this particular embodiment a 532 nm laser is used as the illumination source although this is not required by the present invention.

The collector plastic 308, also referred to as a tape, can be one of several flexible materials, for example, Array Tape™, available from Douglas Scientific of Alexandria, Minn., or other tapes that have been utilized in semiconductor manufacturing or biological applications. Array Tape™ provides reaction wells in customized volumes and formats that are suitable to receive the ejected DNA. In some embodiments, the collector plastic 308 can be moved with respect to the ejection laser beam at higher rates than available using a collector plate as illustrated in FIG. 13, increasing system throughput. One of ordinary skill in the art would recognize many variations, modifications, and alternatives. Registration of the reaction wells on the tape can be performed in order to catalog the locations of the specific ejected DNA.

Light from the illumination laser 322 (a CW laser in this embodiment) passes through a shutter 321 which controls the pulse length of the laser light. Single mode fiberoptic cable is used which gives flexibility to where the laser is placed. With the use of fiber optic couplers (not shown) it is possible to connect a single fiber input to several output fibers. This can reduce cost dramatically and enable affordable multiplexing of optical ejection systems. Multiplexing of DNA ejection systems is preferable since typical second generation sequencers can create up to 100 Gbp of recoverable oligomers (only a small fraction of which are non-redundant and usable). However this necessitates division of sequencing flow cells or the use of multiple flow cells on the same sequencer. After the light passes through the fiber it must be condensed through condensers of increasing focal lengths 318. After the light is focused it enters a cube beam splitter 317 which allows 532 nm light to pass. Afterwards it is directed onto a long wave pass mirrors 305 onto scanning galvonometers 306. These steer the beam to a computer controlled location on the DNA sequencing plate 307, which in this particular embodiment is fixed, although it can be moveable in other embodiments.

Dyes such as Alexa Fluor 532 (Invitrogen, Carlsbad Calif.) can be attached to the DNA so that the ejection can be monitored. This is particularly important with rolony clonal DNA, which is not directly observable like polony beads. As illustrated in FIG. 15, an ejection laser 312 is used to dislodge carrier bound DNA from the plate 307. The ejection laser can range in the UV-A range and higher and like with the illumination laser, a shutter 313 is used to control the length of the laser pulse when CW lasers are used or to modify the pulse properties if a pulsed laser is used and pulse modification is desired. As is also the case with illumination laser, optical fiber and fiber coupling can be used to multiplex the output (not shown here). A beam expander is used to control the size of the beam on the plane of the carrier bound DNA. A half wave plate is used to control the orientation of the transverse wave. The beam is then reflected at 90° off a mirror and enters the beam splitter 305. A small fraction of the light used passes through the long wave pass filter and is directed onto a power meter 304. The power meter allows for readjustment of laser energy to prevent excessive heating (and possible co-ejection of adjacent samples) or insufficient power necessary to enable ejection. The laser travels along the same optical train as the illumination laser to reach the carrier bound DNA on plate 307.

The apparatus also includes a camera 300 to capture the ejection of DNA at a high frame rate, light is transmitted into the camera 300 from a lens 301 and filter 302 to limit observation to a certain wavelength. A stop 303 is positioned between the filter and the mirror to prevent unwanted light from angles not aligned with the sequenced DNA chip. This stop or aperture determines how collimated the beam is which is very important to the appearance of the image plane. A light source (e.g., an LED in the visible range such that it is passable to the long wavelength mirror) is also located below the DNA plate in order to allow for non-fluorescent imaging to attain the proper focus when the DNA is not fluorescently labeled.

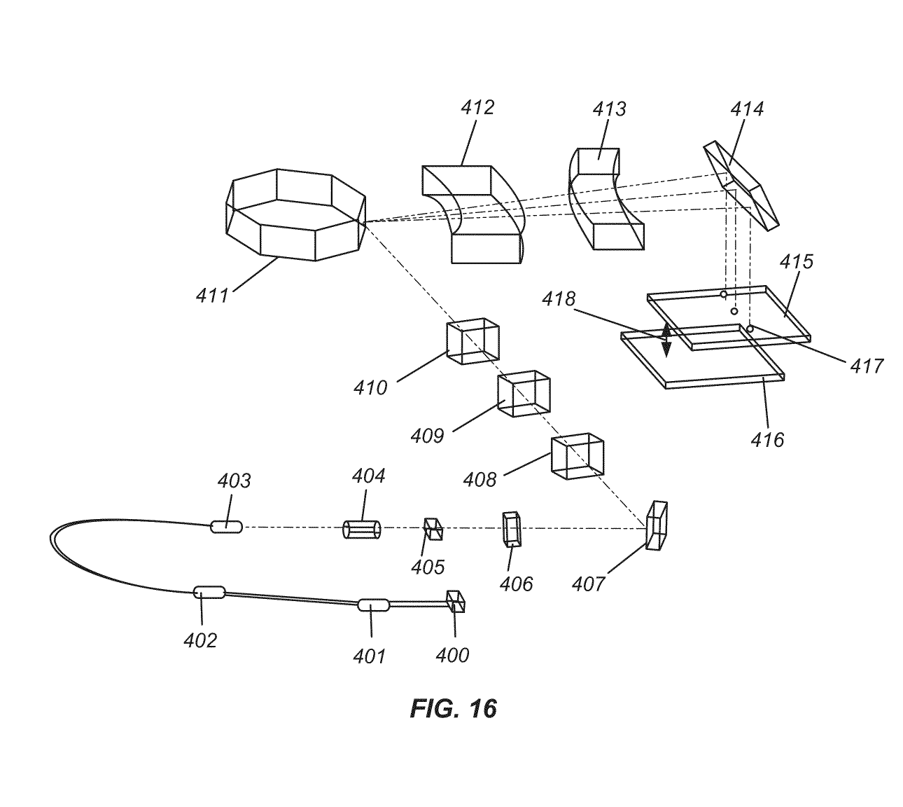

FIG. 16 is a simplified diagram illustrating a polygonal mirror based beam scanning system for ejecting DNA. In this model, a continuous wave laser 400 of a fixed wavelength is used. Light travels onto the DNA sequencing plate 415 by moving though the optical coupling 401 to the first optical fiber towards a connector 402 along a second fiber optic cable towards an optical end 403. Light is then focused through a coupling imaging lens 404, which serves to control the shape of the beam. The beam then passes through an acousto-optic modulator 405, which is used to diffract and shift the wavelength of light acting as a Q switch. This is important in some embodiments since when doing a scanning operation it is desirable to hit selected areas of the beam path to release select DNAs. The beam is then reflected off a mirror 407, which can be partially transmissive in order to measure beam energy and potentially be coupled with a feed back circuit (not shown). Light is then directed towards a series of lenses 408, 409, and 410 after which point it is directed onto a polygonal mirror 411, which rotates in order to scan the beam of light across the scan area. Next the beam passes through two additional lenses 412 and 413, which limit the divergence of the beam off a mirror 414 onto a colony DNA 417 on the DNA plate 415, which rests a fixed distance 418 above the collector plate 416 in this embodiment. The scanning system preferably meets several requirements to allow for proper colony DNA ejection. First the beam must be focused at an energy and resolution sufficient to eject the DNA. This spot size should not change over the course of the scan nor should the beam dwell over any one particular area for an aberrant period of time. The optics should be appropriate for the wavelength and the angle at which the beam hits the DNA plate should be appropriate. Since the scanned spot is not a linear function of the deflection angle in standard scanning lens (requiring the use of complicated scanning algorithms) special scanning lenses 412 and 413 are necessary to provide a flat field by adding a specific amount of distortion.

Although a collector plate 416 is illustrated in FIG. 16, other embodiments can utilize the plastic tape as a collection system as discussed in relation to FIG. 15. One of ordinary skill in the art would recognize many variations, modifications, and alternatives.

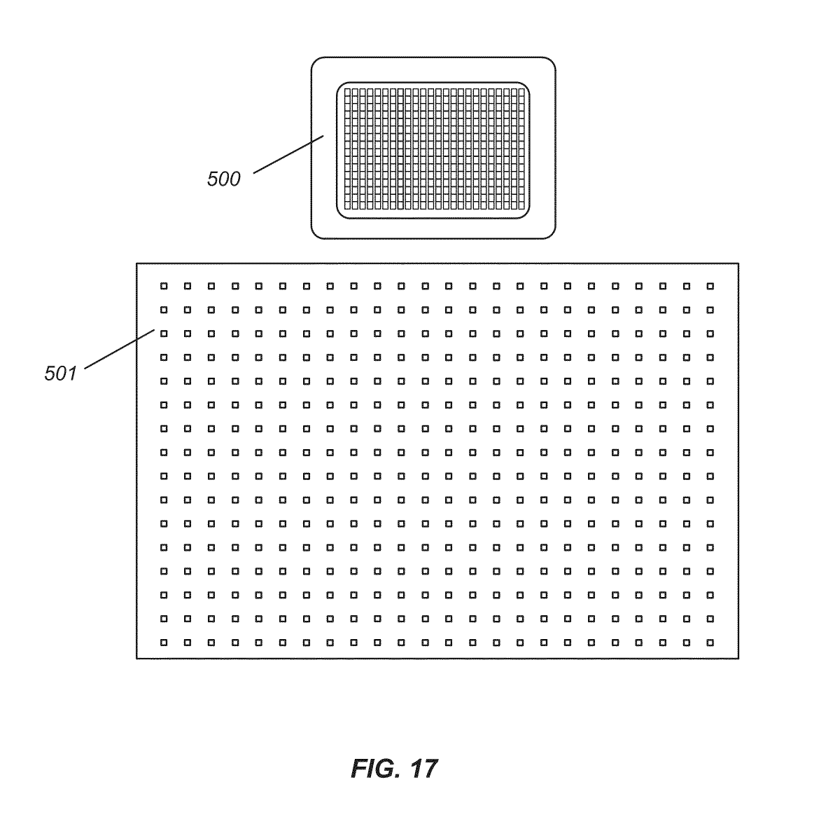

FIG. 17 is a simplified diagram illustrating a sequencing plate in which the plate is divided up into 384 subdivisions 500. To reduce the amount of scanning done by the CCD these surfaces on which the DNA is sequenced are close together 501. However after sequencing these surfaces are spread apart to cover a larger area in line with a 384 collector plate. This can be done by vacuum transport in which 384 vacuum nozzles move and serially release the plates. Because of the redundancy of the sequences a correct version of the sequence originating from each spot on a microarray synthesizer should be found on the plate (if not multiple times). This allows chip to be processed rapidly for each ejection using a F-theta scanning lens or other scanning lens that covers a wide area.

The sequencing plate illustrated in FIG. 17 provides an optional approach in comparison with sequencing processes in which the beads are formed with a random spatial distribution. By performing surface modification to the substrate (e.g., glass), the DNA can be sequenced preferentially at desired locations as illustrated in FIG. 17. Thus, both random distributions of beads as well as patterned arrays are included within the scope of the present invention. The spatial separation between DNA provided by the embodiment illustrated in FIG. 17 can prevent issues that arise when randomly distributed DNA are close enough that the ejection process results in multiple DNA being ejected. As an example, the beads illustrated in FIG. 17 can be ˜1 μm in size with a pitch of ˜1.2 μm, although other geometries can be utilized. For instance, 3 μm beads could be used with up to an extra 20% spacing between beads. One of ordinary skill in the art would recognize many variations, modifications, and alternatives. In some embodiments, the laser spot size is controlled to be smaller than the size of the bead (e.g., 95%, 90%, 85%, 80%, 75%, or the like) in order to provide for accurate control over bead removal. The spatial separation between beads and the dimensions of the laser spot are then suitable for removal of single beads in a predetermined manner.

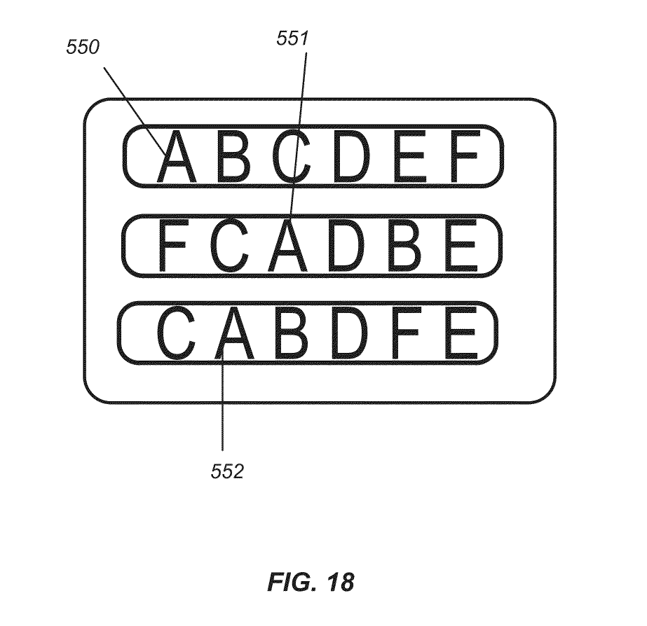

FIG. 18 is a simplified diagram illustrating the redundancy of sequences showing up in different lanes. Each of these lanes may contain up to 150 million features. The number of lanes can match up with the number of rows or columns on the acceptor plate or film. This allows plates or film to be moved rapidly under the sequencing plate while a laser pulses up to 1000 times a second. To ensure amplification by PCR after ejection it is possible to eject the same sequence into the same tube multiple times. It is also possible to eject different overlapping sequences into the same tube to save time on the robotic assembly step. This is similar to the pooled PCR approach in which subpools of overlapping sequences with the same primer region are used to selectively amplify. However in this scenario the assembly is simplified because only a single universal exists for all sequences and because only the sequences to be assembled exist in the subpool. This gets rid of a lot of the off priming effects in which primers amplify a sequence in one of the oligos or when similar sequences prime each other. Usually when synthesizing many genes or genetic circuits one wants to perturb a small space with little variation, so this is much more useful in most cases than ordering a large number of different genes. Thus, embodiments of the present invention make the pooling approach feasible on an industrial scale.

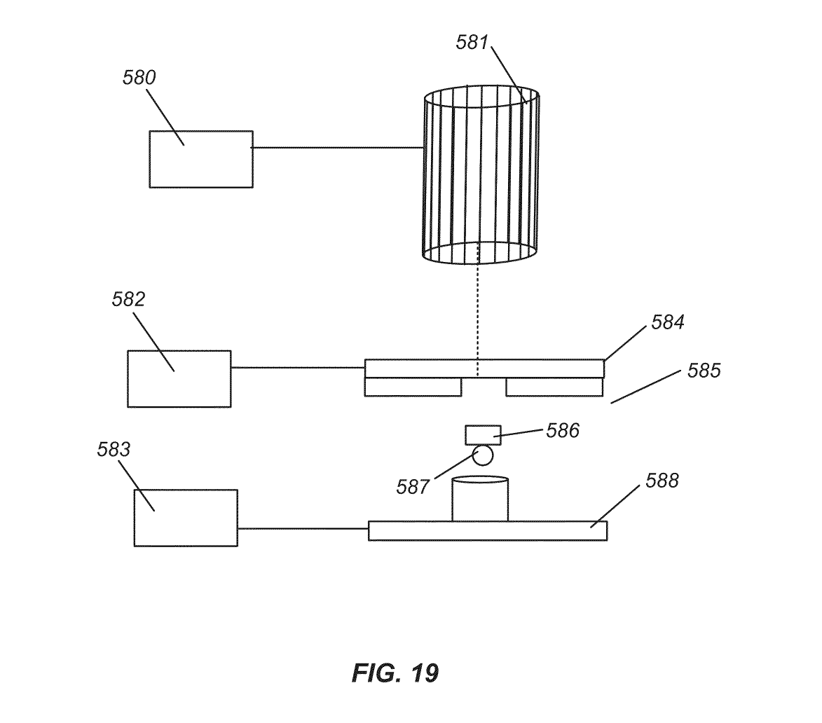

FIG. 19 is a simplified diagram illustrating the use of a sacrificial layer used to absorb the energy from the laser pulse ejecting the DNA via a shockwave or the ejection of the substrate from the backing transparent surface. This laser induced forward transfer event is common to laser direct write commonly used for the printing of electronic components. In this approach, a high repetition rate pulse laser is focused through a transparent support and onto a 5-10 μm matrix. This matrix can be made of a variety of materials. The apparatus used to eject DNA includes a target transparent support made of glass, quartz or a transparent polymer 584. A pulse laser 580 sends laser pulses through an objective 581 into the target substrate 585, which is adjacent to the support. The target substrate 585 can be made of, but is not limited to, polymers including hydrogels (such as gelatin), sol gels, proteins, metals, and the like. Control of the beam position on the plate in this case is controlled by a moving stage 582. Position of the collection plate 588 is also controlled by a moving stage 583 in this embodiment although just one or both of these techniques can be utilized. In this embodiment, a reaction chamber located on collection plate 588 is used to collect the DNA 587 bound to the spall 586.

As illustrated in FIG. 19, the target substrate 585 provides a sacrificial layer attached to the bead. The sacrificial layer absorbs the laser energy and is ablated, providing protection from excess deposition of laser energy in the bead. As illustrated a portion 586 of the sacrificial layer 585 is ejected in response to the deposition of the laser energy. The attachment mechanism between the bead and the sacrificial layer can include one of several attachment chemistries, including, amines, alkane alzide, non-covalent binding using short tethered oligos, and the like.

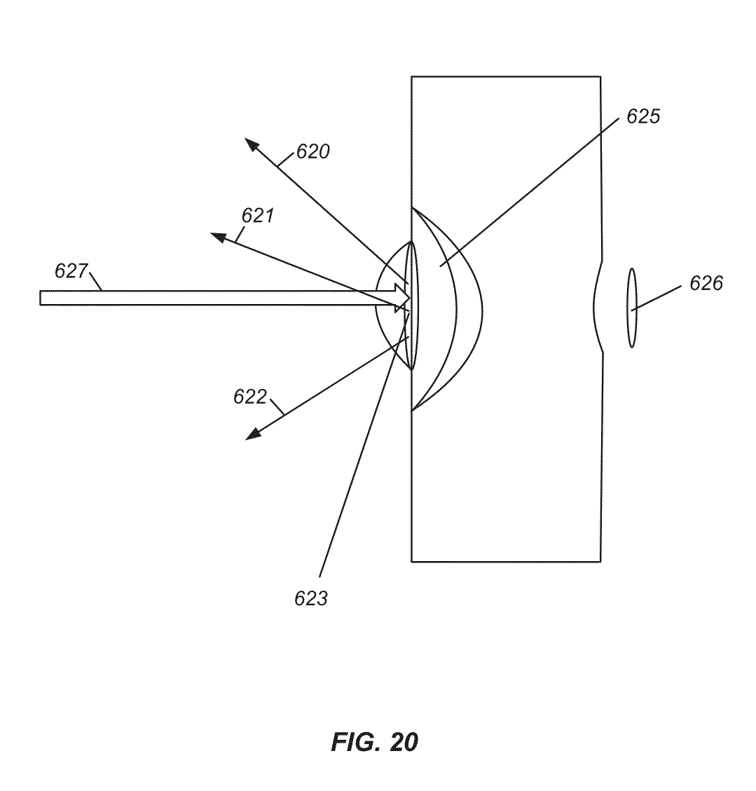

FIG. 20 is a simplified diagram illustrating the laser-solid interaction. The laser beam 627 striking the polymer ejects electrons, ions, plasma 620. The interaction also reflects light 621 and releases vapor 622 leaving behind a crater 623. The energy also dissipates a shockwave 625 that dissipates heat and momentum ejecting the carrier or substrate bound DNA on the spall created by the firing. Spall are the pieces of the matrix material that break off during the spallation event 626. This ejected matrix material (spall) is bound to the DNA either in bead (polony) or substrate bound DNA ball form (rolony). A high energy pulse laser such as an Nd:YAG creates a compressive stress on the substrate and this energy propagates and creates a tensile wave whose properties relate to the tensile strength of the matrix in response to cavitation. The DNA spall is likely traveling at a very high speed in the nanoseconds after the firing and moving at a speed as high as one third the stress wave speed on the material. Longitudinal stress is desired but too much sheer stress or the amount of stress exerted that is coplanar to the material cross section can result in unwanted spallation of neighboring colony DNA.

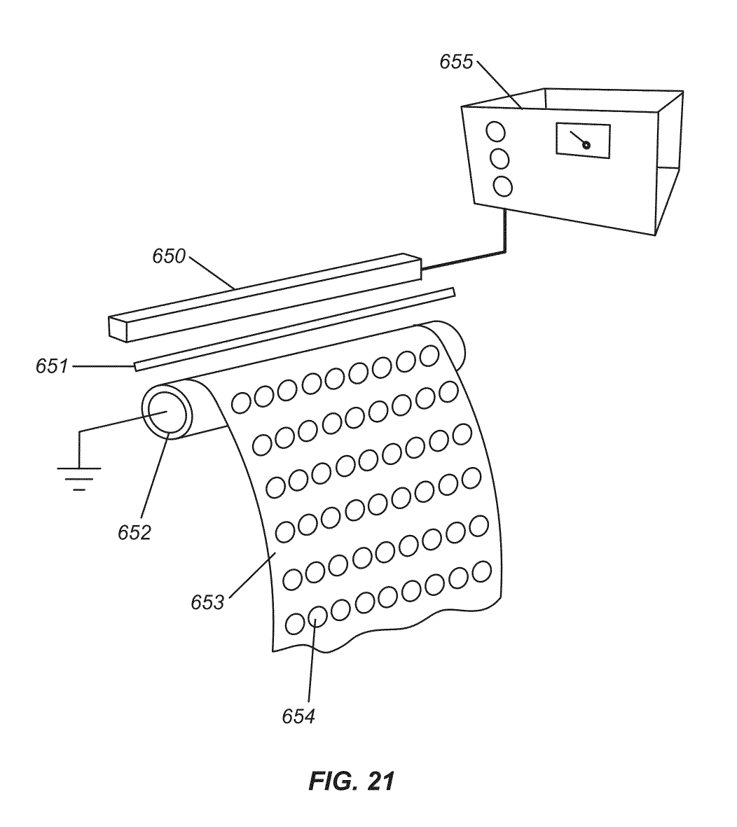

FIG. 21 is a simplified diagram illustrating the treatment of a collector film using corona discharge. The collector film can be a collector plastic as discussed in relation to FIG. 15. Corona treatment uses electric currents to create an ozone generating spark that changes the surface characteristics of many plastics such as polypropylene or polyethylene. In the system illustrated in FIG. 21, a power supply 655 is connected to an electrode 650 that is masked to align with the wells 654 on the DNA receiving film 653 which acts as a dielectric material. A roller 652 acts as the electrical ground so on the whole the device can be thought of as a capacitor. A mask is shown which may be a removable layer to selectively treat wells. Corona treatment can be used to confine reagent to wells to simplify loading or differential charge can be used to direct ejected carrier bound DNA to a dry well for liquid loading post-ejection. Utilizing the surface treatment process illustrated in FIG. 21, the probability of the ejected DNA being contained in a well after ejection is increased. In some embodiments, a charge is localized on wells 654, enabling electrostatic attraction with ejected DNA that possess a charge after ejection.

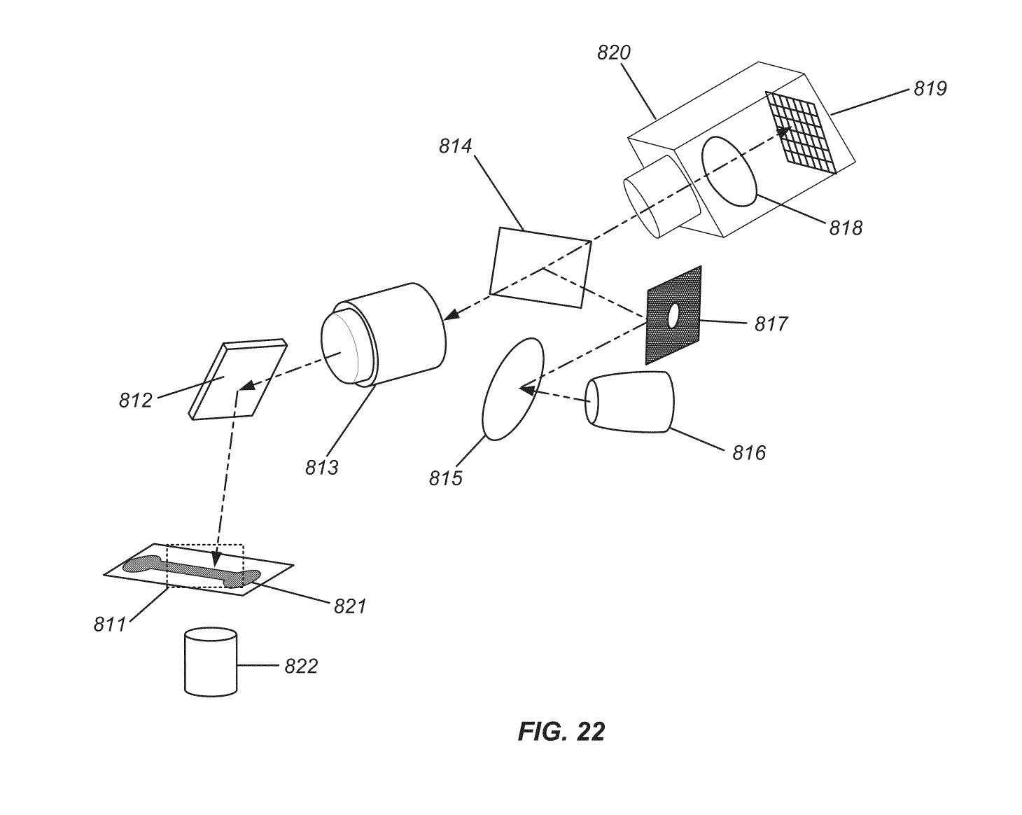

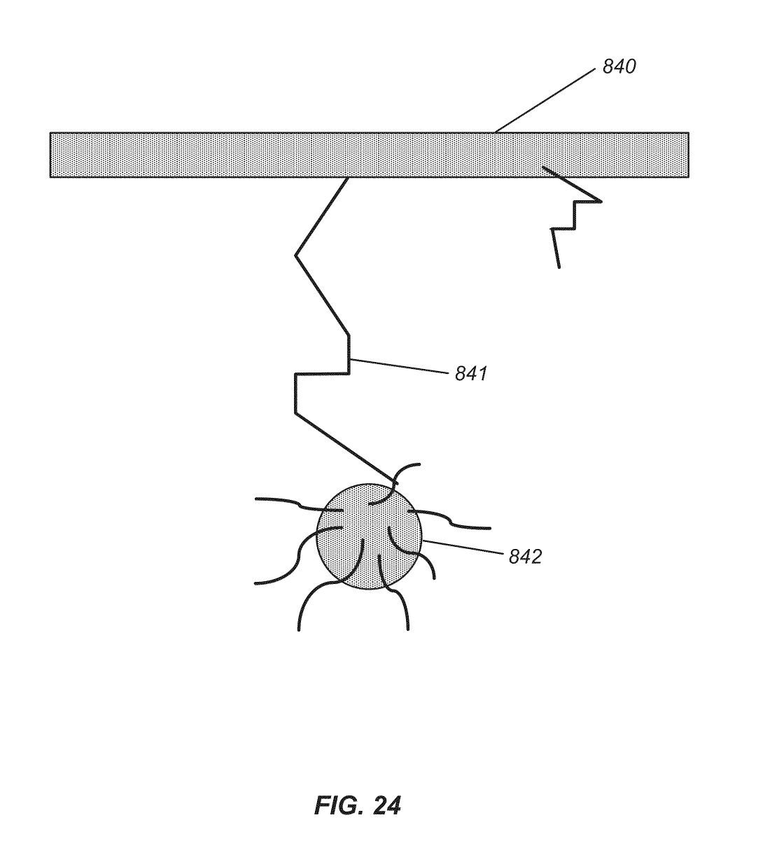

FIG. 22 is a simplified diagram illustrating a digital micromirror setup used to release sequenced DNA from a substrate. Released DNA can either be “polony” or bead bound DNA or “rolony” DNA the product of rolling cycle assembly. 816 shows the source of polarized light a UV source and collimator. The light is passed off a UV mirror 815 to a digital micromirror device 817. The DMD projects a specific image by varying the angle of its electronically controlled micro-mirrors. The light is then projected towards a dichroic mirror 814 which functions as a short pass filter. The dichroic mirror by functioning as a beam splitter allows transmitted light to through to an imaging lens 818, a CCD camera 819, and an imaging sensor 820, which allows imaging of the sample. After redirection by the dichroic mirror collimated light originally from the UV lamp is focused through an objective lens 813 after which point it is redirected by a turning mirror 812 onto the target area 821 of the next generation sequencing chip 11, preferably made of glass. In the case of microfluidic DNA collection a microfluidic chip can be placed on top of the next gen sequencing chip to create a contained environment. As discussed in subsequent figures an electrokinetic concentrator can be integrated into the microfluidic device. The light can activate a photo-cleavable linker to separate the DNA from the substrate. Additional description related to photo-cleavable linkers is provided in relation to FIG. 24.

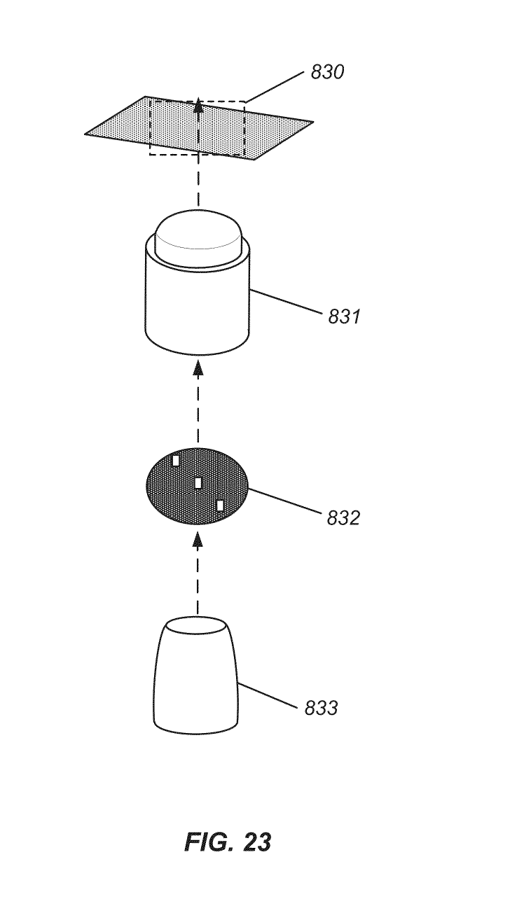

FIG. 23 is a simplified diagram illustrating an alternate liquid crystal display (LCD) based DNA release system in which the LCD mask is used to control the location of the light pattern. Light passes from the light source 833 to an LCD 832. The LCD is computer controlled to allow the passage of light where the DNA sample is to be removed. After selective passage through the LCD, light is focused by an objective 831 onto the clonal DNA to release it from the substrate 830 via severing the photo-cleavable linker and allowing for fluidic collection using a microfluidic chip.

FIG. 24 is a simplified diagram illustrating a polony bead bound DNA 842 attached by a photo-cleavable (PC) linker 841 to a substrate 840. The photo-cleavable linker can be PC should be sensitive to UV light at a wavelength not damaging to the DNA. Many PC linker options exist and are suitable for use with embodiments of the present invention.

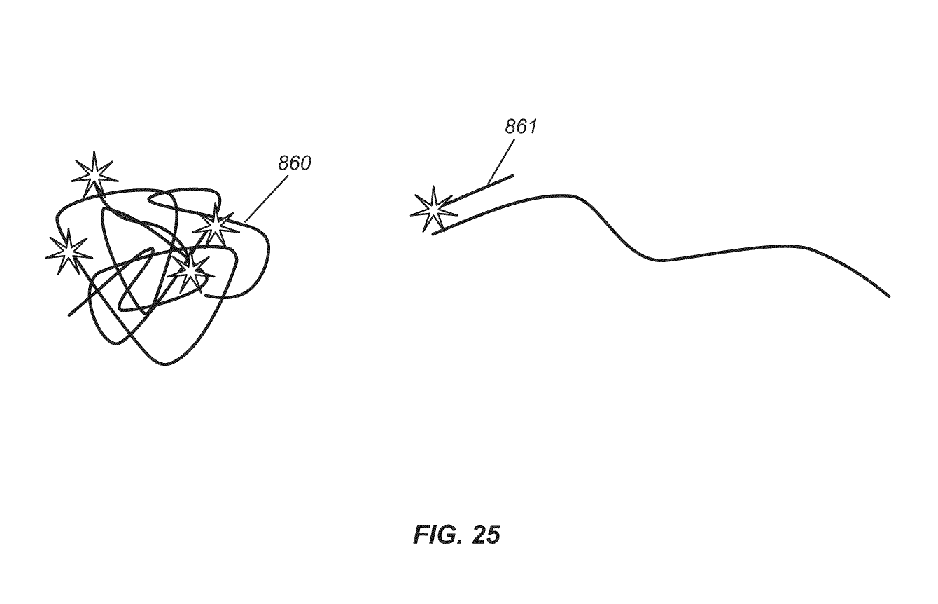

FIG. 25 is a simplified diagram illustrating a fluorescence labeled rolony 860 and the universal primer 861 with 5′ fluorophore used to label it. A rolony is created a through rolling circle amplification of circularized DNA. Rolling cycle PCR produces multiple single stranded copies in a head to tail series called a concatemer. Like the polony based method can create errors, which may not be discovered until resequencing if the errors do not form a substantial portion of the rolony. By substantial, not enough to be detected as heterogeneous during sequencing.

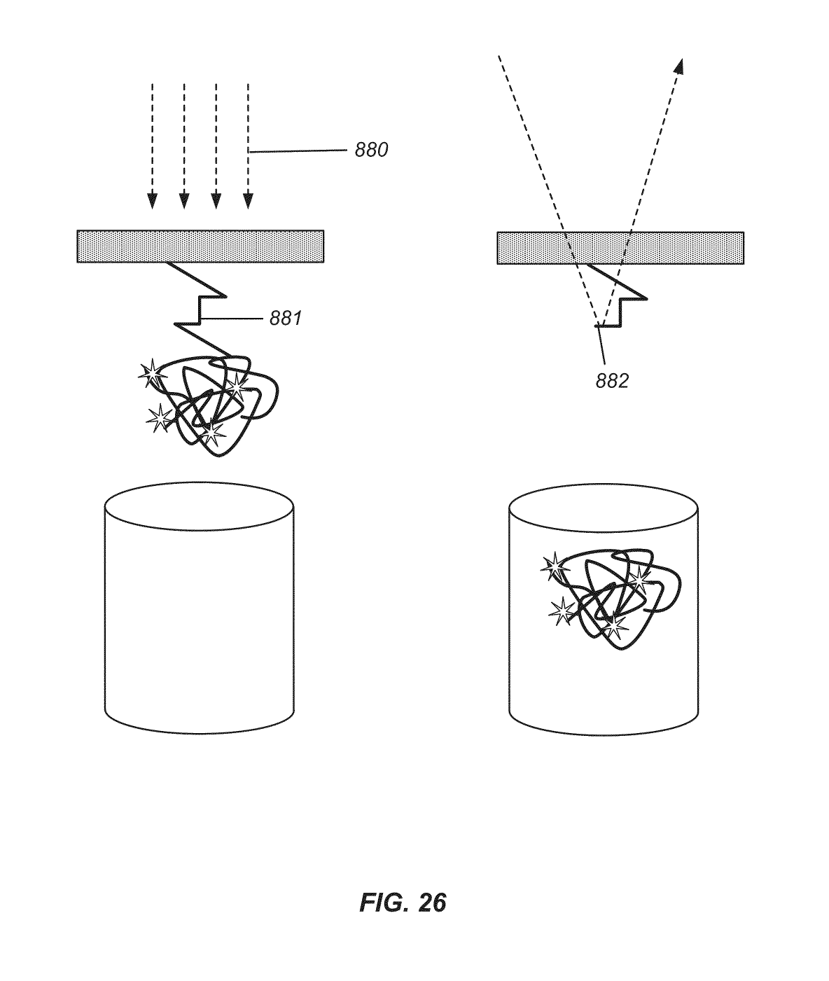

FIG. 26 is a simplified diagram illustrating dry release of rolony DNA 881 by UV light deflected by a digital micromirror device 880. It also shows optical observation of the release. The fluorescence hybridization is done so that the micron sized DNA ball can be visualized. Unlike laser based bead ejection, the photocleavable activity, as well as the strength of the attachment will vary. Since the coupling efficiency of the DNA to the substrate cannot be known a priori it is important to have physical confirmation of the release event. Otherwise there will not be amplified DNA and this will obviously interfere with the assembly. Furthermore running a gel after amplification and purification is time consuming. Physical observation of DNA release is much more preferable then downstream detection approaches.

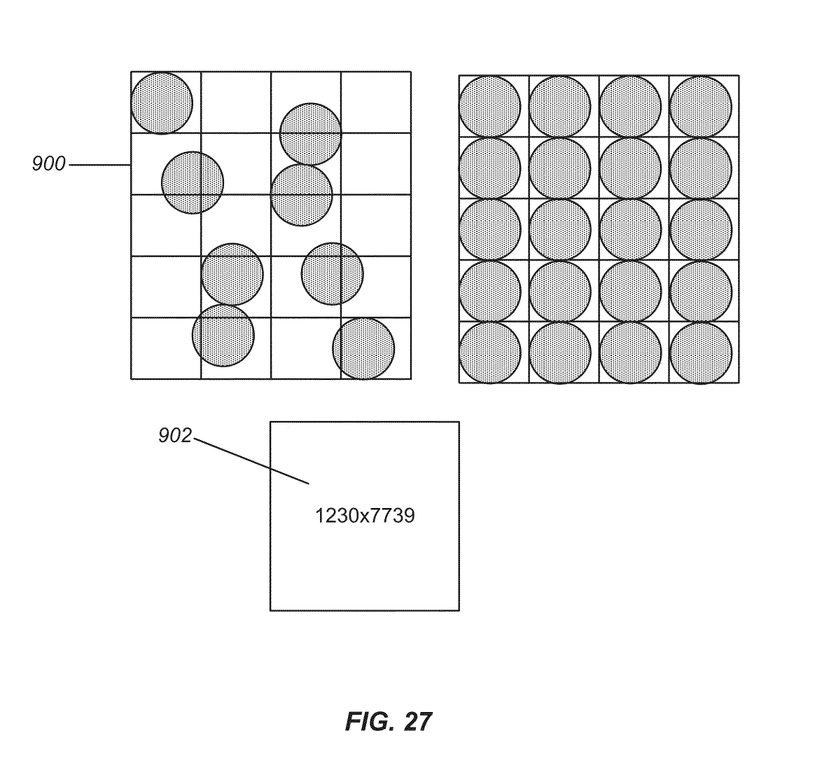

FIG. 27 is a simplified diagram illustrating the use of a grid in order to more easily define relative positions of beads on the sequencing chip. Rolony/polony DNAs are affixed to the sequencing chip prior to sequencing. 900 shows a grid on a non patterned flow cell. Without a patterned flow cell it is likely that beads will congregate and more than a single polony/rolony will appear within a certain grid. More troublesome than that is that this may make it difficult to eject one of the polony/rolonies without accidently releasing the neighbor polony/rolony. A patterned flow cell can prevent this problem, at least in the case of polonies 901. Rolonies may present more difficulties because of the size and lack of a very defined structure. A flow cell would also overcome the need to drastically decrease sequencing density to ensure sufficient clonal DNA isolation. Micropatterning of individual wells or polony containers might be useful for confirming image processing/algorithm based mapping 902.

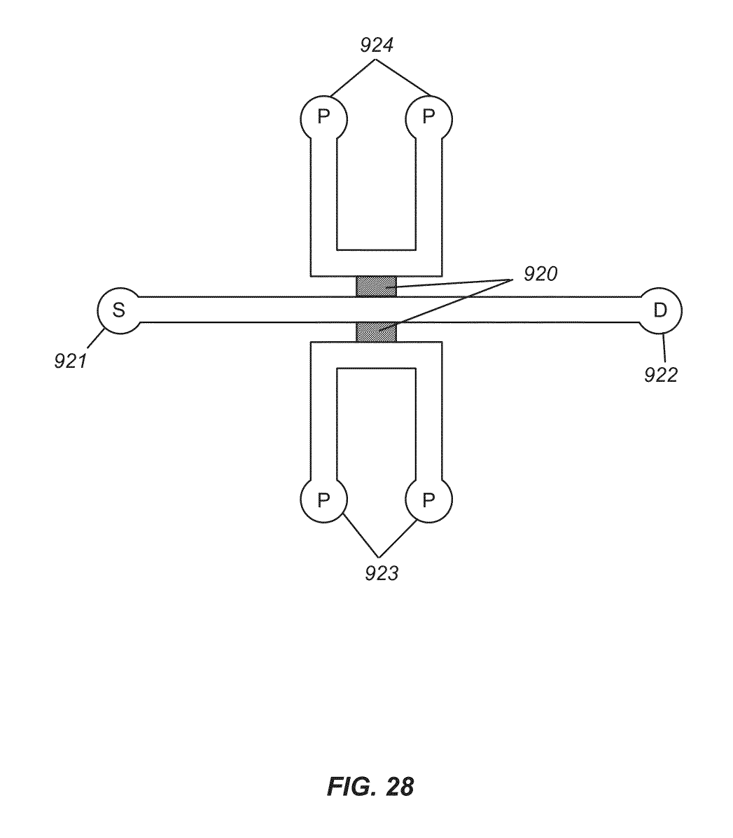

FIG. 28 is a simplified diagram illustrating an electrokinetic DNA concentrator for use with aqueous laser or digital micromirror based DNA release. This DNA concentrator can be used with sample preconcentration exploits various sample characteristics, including electric charge, size, mobility, and affinity. DNA has the unique property of having a uniform charge to size ratio, making charge selective preconcentration efficient. Among the preconcentration techniques using the electric property of DNA, the electrokinetic trapping technique has received much attention due to its high sensitivity and easy miniaturization. Electrokinetic trapping usually utilizes a charged nanochannel (or nanoporous membrane) between microchannels, allowing for enrichment of counterions along the structure while excluding coions. The local electric field gradient and concentration polarization are induced when an electric field is applied across the charged nanochannel so that sample preconcentration can be achieved. The electrokinetic trapping technique does not suffer from overall device size, since the preconcentration occurs in the immediate proximity of the ion depleted boundary, and has shown a high efficiency for concentrating charged biomolecules. However, the conventional fabrication process to incorporate an ion-permselective membrane into a microfluidic device involves complex steps. Although details regarding electrokinetic trapping can be found elsewhere, a brief description will be given here. When P reservoirs 924, 923 are grounded to 0 V, cations are selectively extracted through the negatively charged hydrogel, and anions expelled from the area near the negatively charged hydrogel 920. As a result, an ion-depleted region develops between the two hydrogel plugs. Along the ion-depleted region, the electric resistance and corresponding electric field substantially increase. Therefore, electrophoretic force (EP) is locally strengthened at the ion-depletion region. Near the ion-depletion region, the direction of anion EP is the opposite of the EOF of the main channel, thus anions entering the ion-depleted region experience an enhanced EP that drives back toward the sample reservoir. Consequently, anions are stacked to the left of the ion-depletion boundary where the main channel flow rate and EP balances each other. To trap of the DNA oligonucleotides in 0.1×TE buffer mixture are flowed into the main channel (S-D) 921, 922. At this point 40 V at the sample reservoir (S), 5 V at the drain reservoir (D), and 0 V at the polymer reservoirs (P). If the DNA is fluorescently labeled, at time zero, before the voltage application, the fluorescence signal is very weak. However, a strong fluorescent signal will be detected at the left side of the ion-depleted region after the application of voltage. The fluorescence intensity increases linearly before leveling off and saturating. The result reveals that DNA can be rapidly and effectively concentrated using the digital micromirror fabricated charged nanoporous hydrogel system for visualization of DNA released from a sequencing chip into a microfluidic channel. Additional description related to electrokinetic DNA concentrators is provided in commonly assigned U.S. patent application Ser. No. 13/725,300, filed on Dec. 21, 2012, the disclosure of which is hereby incorporated by reference in its entirety for all purposes.

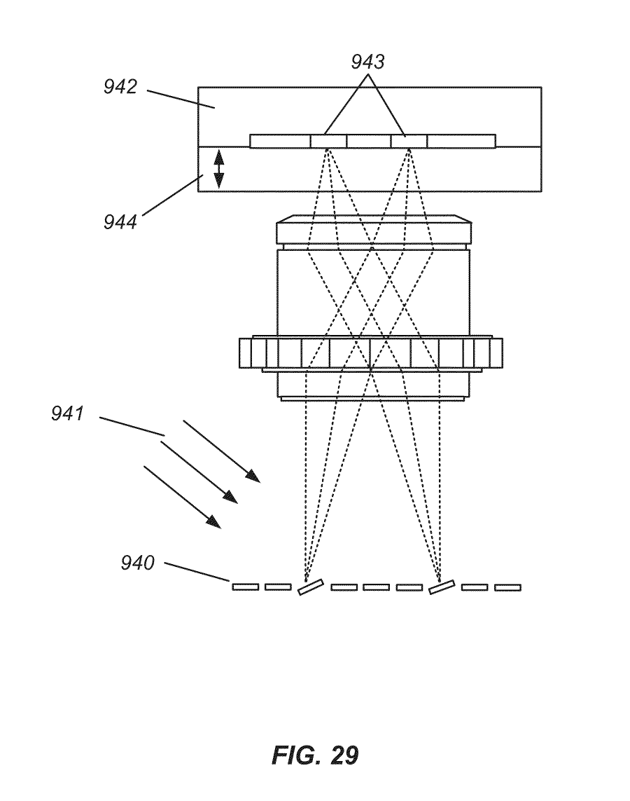

FIG. 29 is a simplified diagram illustrating how a digital micromirror can be used to fabricate charged nanoporous hydrogel necessary to construct the electrokinetic concentrator. By using a DMD system a, computer-controlled spatial light modulator dynamically generates arbitrary pattern of UV light, reducing complex polymerization process by doing away with the necessity of physical photomasks and mask alignment. The OFML system has the unique advantage in fabricating small polymer structure through a thick substrate because the objective lens can focus the pattern at a desired depth. In addition, appropriate focal depth choice of the objective lens can ensure a vertical polymer shape in the microchannel. The UV light 943, DMD 940, objective, substrate height 944, microchannel 942, and polymerized nanoporous hydrogel 943 are shown.

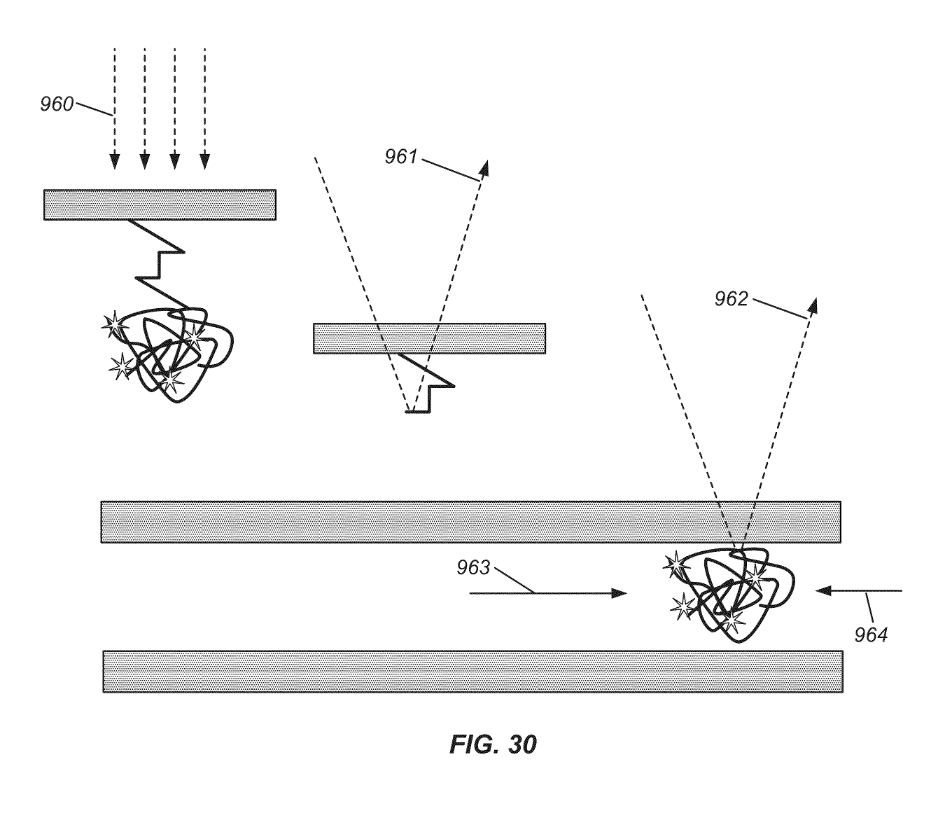

FIG. 30 is a simplified diagram illustrating UV light release of a rolony bound to a substrate with a photocleavable linker that is visualized after release of DNA into the microfluidic channel. In this way it should be possible to monitor the release and trapping of rolony DNA in the electrokinetic concentrator 962.

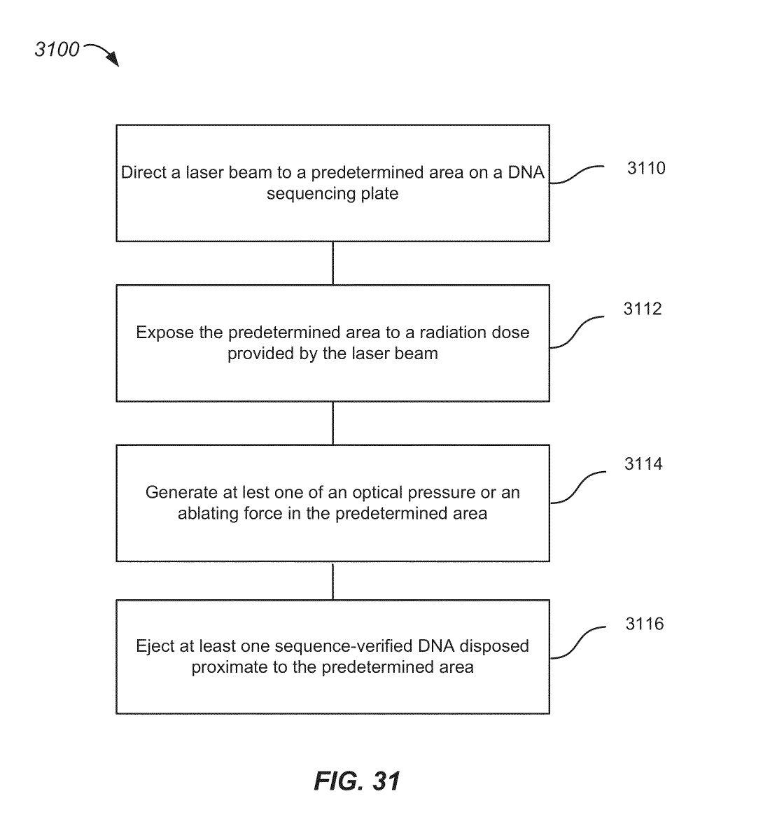

FIG. 31 is a simplified flowchart illustrating a method of recovering sequence verified DNA according to an embodiment of the present invention. The method 3100 utilizes a DNA sequencing plate and includes directing a laser beam to impinge on a selected area on the DNA sequencing plate (3110). The DNA sequencing plate contains a plurality of sequence-verified DNA disposed thereon. Directing the laser beam can utilize various techniques including steering the laser beam using a mirror, a prism, a diffraction grating, a phased-array optics device, a microelectrical mechanical systems (MEMS), galvanometer scanning mirrors, one or more F-Theta scanning lenses, Riley prisms, a polygonal-mirror-based beam scanning system, a digital micromirror device (DMD), a liquid crystal display (LCD) mask, combinations thereof, or the like.

The method also includes exposing the selected area to a radiation dosage provided by the laser beam (3112) to generate at least one of an optical pressure or an ablating force in the selected area (3114), thereby ejecting at least one sequence-verified DNA disposed proximate to the selected area (3116). As an example, the laser beam can be a pulsed laser beam characterized by a wavelength ranging from about 157 nm to about 10,600 nm. Using pulsed laser beams, the laser beam can be characterized by a pulse width ranging from about 10⁻¹² second to about 10⁻⁸ second, and a pulse repetition rate greater than about 100 kHz.

In some implementations, the he DNA sequencing plate includes a sacrificial layer disposed adjacent the plurality of sequence-verified DNAs. The sacrificial layer absorbs the radiation dosage provided by the laser beam and protects the DNA during ejection. The sacrificial layer can include a polymer film and the DNA sequencing plate can also include a partially transparent metal film disposed underneath the sacrificial layer.

In other embodiments, a continuous wave laser beam is utilized, with a wavelength of about one of 193 nm, 248 nm, 266 nm, or 355 nm, a fluence ranging from about 1 nJ/cm² to about 1 J/cm², from about 0.1 mJ/cm² to 1 J/cm², or from about 0.1 mJ/cm² to 1 J/cm². The DNA sequencing plate can be positioned on a first stage aligned with respect to the laser beam. At least one ejected sequence-verified DNA is collected by a collection plate (e.g., containing a plurality of wells) positioned on a second stage aligned with respect to the first stage. As an alternative, the at least one ejected sequence-verified DNA can be collected by a flexible film including a plurality of wells.

In an embodiment, The method further includes performing a parallel single molecule synthesis process to form the plurality of sequence-verified DNAs on the DNA sequencing plate. The at least one sequence-verified DNA can include a plurality of sequence-verified DNAs with overlapping sequences. The plurality of sequence-verified DNAs can then be collected in a single receptacle for pooled polymerase chain reaction (PCR). In another embodiment, the at least one sequence-verified DNA comprises a plurality of sequence-verified DNAs disposed in a plurality of lanes and the plurality of lanes include redundant sequences.

The DNA sequencing plate further includes a electrokinetic concentrator in one embodiment. As described herein the DNA sequencing plate can include an ejection plate that is part of a two-part flow cell. The ejection plate can be removed from a chemistry substrate and then placed into an optical system providing optical access to the impingement surface of the ejection plate.

It should be appreciated that the specific steps illustrated in FIG. 31 provide a particular method of recovering sequence verified DNA according to an embodiment of the present invention. Other sequences of steps may also be performed according to alternative embodiments. For example, alternative embodiments of the present invention may perform the steps outlined above in a different order. Moreover, the individual steps illustrated in FIG. 31 may include multiple sub-steps that may be performed in various sequences as appropriate to the individual step. Furthermore, additional steps may be added or removed depending on the particular applications. One of ordinary skill in the art would recognize many variations, modifications, and alternatives.

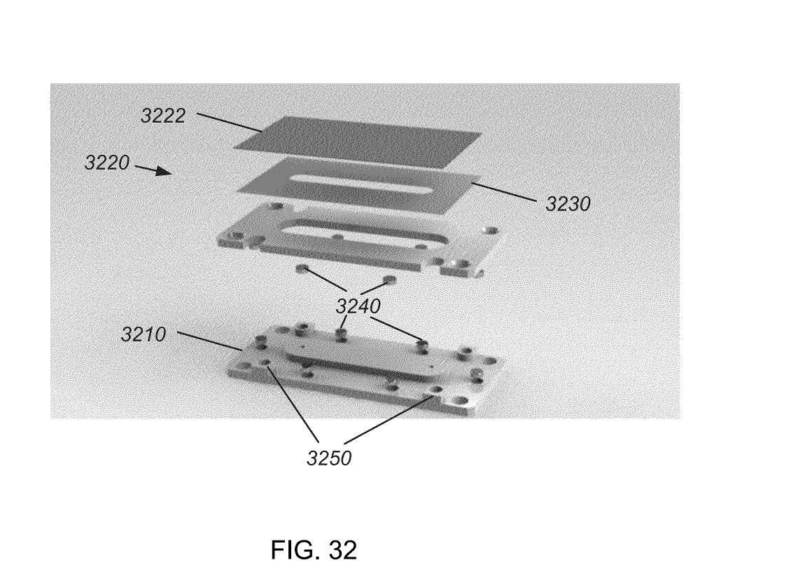

FIG. 32 is a simplified schematic diagram of a two-part flow cell according to an embodiment of the present invention. The two-part flow cell includes a substrate element 3210 (which can also be referred to as a fluidic substrate) and a bead attach element 3220 (which can also be referred to as an ejection substrate or ejection plate) and includes several sub-elements. The bead attach element 3220 provides a transparent portion 3222 (at wavelengths used for ejection) suitable for passing the light (e.g., laser light) used to eject the DNA beads. For visible wavelengths, glass materials are suitable.

The two-part flow cell is used to perform performing chemistry to sequence the DNA, which are attached to the beads on the bead attach element 3220. After sequencing, the two-part flow cell is disassembled, providing optical access to the interior portion 3230 of the bead attach element 3220. The bead attach element can then be mounted in a suitable carrier for positioning and movement with respect to the laser beam used to eject the DNA beads. Referring to FIG. 13, the bead attach element can be mounted as illustrated by DNA chip 257, with the interior portion 3230 facing up in order to receive the laser beam. Impingement of the laser beam produces ejection of the DNA as described more fully throughout the present specification.

During chemistry operations, the substrate element 3210 and the bead attach element 3220 are held together with magnets 3240 in some implementations. Fluid ports 3250 are provided on the substrate element 3210 to provide for liquid flow in and out of the two-part flow cell.

According to embodiments of the present invention, the DNA sequencing plate, which is the bead attach element of the two-part flow cell, includes a sacrificial layer (e.g., a polymer) to which the beads are attached. After disassembly, the DNA sequencing plate provides optical access to the polymer layer for DNA ejection.

The foregoing description of the exemplary embodiments has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

It should be appreciated that the methods and systems described herein can be used in an interchangeable manner, with components and techniques utilized in one implementation also utilized in alternative implementations. One of ordinary skill in the art would recognize many variations, modifications, and alternatives.

It is also understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A method of retrieving sequence-verified deoxyribonucleic acid (DNA) from a DNA sequencing plate, the method comprising:
directing a laser beam to impinge on a predetermined area on the DNA sequencing plate, wherein the DNA sequencing plate contains a plurality of sequence-verified DNA disposed thereon; and
exposing the predetermined area to a radiation dosage provided by the laser beam to generate at least one of an optical pressure or an ablating force in the predetermined area, thereby ejecting at least one sequence-verified DNA disposed proximate to the predetermined area.

2. The method of claim 1 wherein the laser beam is a pulsed laser beam.

3. The method of claim 2 wherein the laser beam is characterized by a wavelength ranging from about 157 nm to about 10,600 nm.

4. The method of claim 2 wherein the laser beam is characterized by a pulse width ranging from about 10⁻¹² second to about 10⁻⁸ second, and a pulse repetition rate greater than about 100 kHz.

5. The method of claim 1 wherein the laser beam is a continuous wave laser beam.

6. The method of claim 5 wherein the laser beam is characterized by a wavelength of about one of 193 nm, 248 nm, 266 nm, or 355 nm.

7. The method of claim 5 wherein the laser beam is characterized by a fluence ranging from about 1 nJ/cm² to about 1,000 mJ/cm².

8. The method of claim 1 wherein directing a laser beam comprises steering the laser beam using at least one of a mirror, a prism, a diffraction grating, a phased-array optics device, or a microelectrical mechanical systems (MEMS).

9. The method of claim 1 wherein directing a laser beam comprises steering the laser beam using at least one of galvanometer scanning mirrors, one or more F-Theta scanning lenses, Riley prisms, or a polygonal-mirror-based beam scanning system.

10. The method of claim 1 further comprising performing a parallel single molecule synthesis process to form the plurality of sequence-verified DNAs on the DNA sequencing plate.

11. The method of claim 1 wherein the DNA sequencing plate is positioned on a first stage aligned with respect to the laser beam.

12. The method of claim 1 wherein the at least one ejected sequence-verified DNA is collected by a collection plate positioned on a second stage aligned with respect to the first stage.

13. The method of claim 12 wherein the collection plate comprises a plurality of wells.

14. The method of claim 1 wherein the at least one ejected sequence-verified DNA is collected by a flexible film comprising a plurality of wells.

15. The method of claim 1 wherein the DNA sequencing plate comprises a sacrificial layer disposed underneath the plurality of sequence-verified DNAs, wherein the sacrificial layer absorbs the radiation dosage provided by the laser beam.

16. The method of claim 15 wherein the sacrificial layer comprises a polymer film.

17. The method of claim 15 wherein the DNA sequencing plate further comprises a partially transparent metal film disposed underneath the sacrificial layer.

18. The method of claim 1 wherein the at least one sequence-verified DNA comprises a plurality of sequence-verified DNAs with overlapping sequences, and wherein the plurality of sequence-verified DNAs are collected by a same receptacle for pooled polymerase chain reaction (PCR).

19. The method of claim 1 wherein the at least one sequence-verified DNA comprises a plurality of sequence-verified DNAs disposed in a plurality of lanes, and wherein the plurality of lanes include redundant sequences.

20. The method of claim 1 wherein the DNA sequencing plate further comprises a electrokinetic concentrator.

21. The method of claim 1 wherein the DNA sequencing plate comprises an ejection plate of a two-part flow cell.

22. A system for retrieving sequence-verified deoxyribonucleic acid (DNA), the system comprising:
a laser source;
a DNA sequencing plate containing the sequence-verified DNA disposed thereon;
optical elements disposed along an optical path between the laser source and the DNA sequencing plate; and
a collection substrate adjacent the DNA sequencing plate.

23. The system of claim 22 wherein the laser source comprises a UV laser.

24. The system of claim 22 wherein the DNA sequencing plate comprises a portion of a two-part flow cell.

25. The system of claim 22 wherein the optical elements comprise a raster scanning system.

26. The system of claim 22 further comprising a moveable stage supporting the DNA sequencing plate.

27. The system of claim 26 further comprising a processor operable to control motion of the moveable stage.

28. The system of claim 22 wherein the collection substrate comprises a multi-well plate.

29. The system of claim 22 wherein the collection substrate comprises a flexible tape.
